(19) United States
(12) Patent Application Publication
(10) Pub. No.: US 2014/0235850 A1
(43) Pub. Date: Aug. 21, 2014

(54) SYNTHESIS OF HMO CORE STRUCTURES

(71) Applicant: Glycom A/S, Kongens Lyngby (DK)

(72) Inventors: Imre Kovács, Debrecen (HU); István Bajza, Debrecen (HU); Markus Hederos, Svedala (SE); Gyula Dekany, Queensland (AU); Sándor Demkó, Debrecen (HU); Nikolay Khanzhin, Humlebaek (DK)

(21) Appl. No.: 14/348,334

(22) PCT Filed: Oct. 1, 2012
(86) PCT No.: PCT/DK2012/050368

§ 371 Date: Mar. 28, 2014

(30) Foreign Application Priority Data
Sep. 30, 2011 (DK) PA2011 70538

(51) Int. Cl. C07H 1/00; C07H 3/04; C07H 3/06

(52) U.S. Cl. CPC C07H 1/00; C07H 3/06; C07H 3/04
USPC 536/123.13; 536/123.1

(57) ABSTRACT

The invention relates to a method for making precursors of HMO core structures comprising a step of reacting an N-acetyllactosamine or lacto-N-biose derivative donor with a lactose or N-acetyllactosamine derivative acceptor, wherein the donor is an oxazoline donor.

### FIELD OF THE INVENTION

The present invention relates to a general method for synthesizing precursors of human milk oligosaccharides (“HMOs”) and, via the precursors, producing Galpβ1-4GlcNAcpβ1-3Galpβ1-4Glc (lacto-N-neotetraose, LNnT) and Galpβ1-3GlcNAcpβ1-3Galpβ1-4Glc (lacto-N-tetraose, LNT), as well as other HMO core oligosaccharide structures, preferably lacto-N-neohexaose (LNnH) and para-lacto-N-neohexaose (para-LNnH) structures, especially in

### BACKGROUND OF THE INVENTION

During the past decade, interest in synthesizing and commercializing HMOs has been increasing steadily. The importance of HMOs has been directly linked to their unique biological activities in humans, such as their antibacterial and antiviral activities and their immune system- and cognitive development-enhancing activities.

The tetrasaccharides Galpβ1-4GlcNAcpβ1-3Galpβ1-4Glc (lacto-N-neotetraose, LNnT) and Galpβ1-3GlcNAcpβ1-3Galpβ1-4Glc (lacto-N-tetraose, LNT) are two of the oligosaccharides occurring in human milk [LNnT: Kuhn et al. Chem. Ber. 1962, 95, 513 and 518, Kobata Methods Enzymol. 1972, 28, 262; LNT: Kuhn et al. Chem. Ber. 1953, 86, 827].

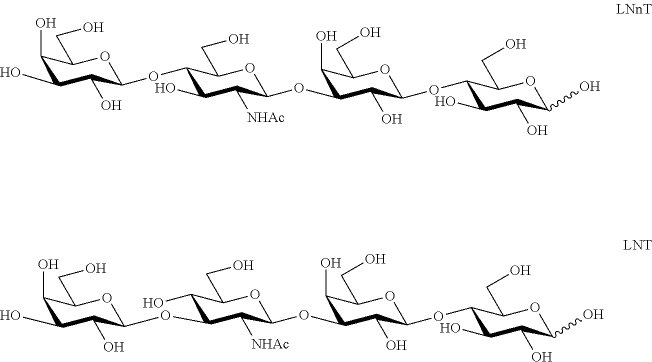

Both LNnT and LNT act as bacterial receptors for pneumococci, and both have been found useful in the recognition of the acceptor specificity of glycosyltransferases, the substrate specificity of glycosidases, and the structure of antigenic determinants. They also represent core structural elements of other HMOs, as well as of other, more complex oligosaccharide cores of glycolipids and glycoproteins having physiological activities in humans.

The core oligosaccharide structures of HMOs are built up from lactose and N-acetyllactosamine and/or lacto-N-biose disaccharide blocks and can be sialylated and/or fucosylated. In each core structure, lactose is at the reducing end. To date, 13 HMO core structures have been proposed (T. Urashima et al.: Milk Oligosaccharides, Nova Biomedical Books, N.Y., 2011):

TABLE 1
13 different core structures of human milk oligosaccharides (HMOs)

| No | Core name | Core structure |
|---|---|---|
| 1 | lactose (Lac) | Galβ1-4Glc |
| 2 | lacto-N-tetraose (LNT) | Galβ1-3GlcNAcβ1-3Galβ1-4Glc |
| 3 | lacto-N-neotetraose (LNnT) | Galβ1-4GlcNAcβ1-3Galβ1-4Glc |
| 4 | lacto-N-hexaose (LNH) | Galβ1-3GlcNAcβ1-3(Galβ1-4GlcNAcβ1-6)Galβ1- |
| | | 4Glc |
| 5 | lacto-N-neohexaose (LNnH) | Galβ1-4GlcNAcβ1-3(Galβ1-4GlcNAcβ1-6)Galβ1- |
| | | 4Glc |
| 6 | para-lacto-N-hexaose (para-LNH) | Galβ1-3GlcNAcβ1-3Galβ1-4GlcNAcβ1-3Galβ1- |
| | | 4Glc |
| 7 | para-lacto-N-neohexaose (para- | Galβ1-4GlcNAcβ1-3Galβ1-4GlcNAcβ1-3Galβ1- |
| | LNnH) | 4Glc |
| 8 | lacto-N-octaose (LNO) | Galβ1-3GlcNAcβ1-3(Galβ1-4GlcNAcβ1-3Galβ1- |
| | | 4GlcNAcβ1-6)Galβ1-4Glc |
| 9 | lacto-N-neooctaose (LNnO) | Galβ1-4GlcNAcβ1-3(Galβ1-3GlcNAcβ1-3Galβ1- |
| | | 4GlcNAcβ1-6)Galβ1-4Glc |
| 10 | iso-lacto-N-octaose (iso-LNO) | Galβ1-3GlcNAcβ1-3(Galβ1-3GlcNAcβ1-3Galβ1- |
| | | 4GlcNAcβ1-6)Galβ1-4Glc |
| 11 | para-lacto-N-octaose (para-LNO) | Galβ1-3GlcNAcβ1-3Galβ1-4GlcNAcβ1-3Galβ1- |
| | | 4GlcNAcβ1-3Galβ1-4Glc |
| 12 | lacto-N-neodecaose (LNnD) | Galβ1-3GlcNAcβ1-3[Galβ1-4GlcNAcβ1-3(Galβ1- |
| | | 4GlcNAcβ1-6)Galβ1-4GlcNAcβ1-6]Galβ1-4Glc |
| 13 | lacto-N-decaose (LND) | Galβ1-3GlcNAcβ1-3[Galβ1-3GlcNAcβ1-3(Galβ1- |
| | | 4GlcNAcβ1-6)Galβ1-4GlcNAcβ1-6]Galβ1-4Glc |

To date, large quantities of LNnT, LNT and related core structures have not been available from known isolation, biotechnology and chemical synthesis methodologies. The isolation of LNnT, LNT and elongated core structures from human milk has been rather difficult even in milligram quantities due to the presence of a large number of similar oligosaccharides in human milk.

Chemical syntheses of HMO core structures including LNnT and LNT have required many reaction steps, protecting group manipulations and chromatographic purifications and provided only poor or modest yields of milligram quantities. Hence, chemical syntheses have not been considered attractive for large scale production.

A key step in prior chemical syntheses of tetrasaccharides like LNnT, LNT, and larger HMO core oligosaccharides has been a glycosylation reaction, coupling a galactosyl oligosaccharide acceptor, like a lactose acceptor derivative, with a disaccharide N-acetyl lactosamine donor or a disaccharide lacto-N-biose donor, and then optionally transforming the resulting tetrasaccharide into a derivative that can accept a further disaccharide N-acetyl lactosamine/lacto-N-biose donor. Disaccharide glucosaminyl derivatives have been considered suitable donors when activated on their anomeric centres by leaving groups, such as trichloroacetimidate, chloride or a thiophenyl group, and have had protected amino groups in the form of e.g., phthalyl, trichloroacetyl or dimethylmaleoyl groups. [Paulsen et al. Carbohydr. Res. 1987, 169, 105; Aly et al. ibid. 1999, 316, 121; Aly et al. Eur. J. Org. Chem. 2000, 319; Ponpipom et al. Tetrahedron Lett. 1978, 20, 1717; Malleron et al. Carbohydr. Res. 2008, 343, 970; WO 2011/100980 A1].

Oxazoline monosaccharide donors have also been used with promoters to provide direct and stereoselective glycosylation to monosaccharide acceptors [Toshima: 5.3 Miscellaneous Glycosyl Donors, 5.3.11 Oxazoline In: Handbook of Chemical Glycosylation (Ed.: Demchenko), Wiley, 2008, pp. 457-458; Xia et al. Bioorg. Med. Chem. Lett. 1999, 9, 2941]. Hence, the use of oxazoline disaccharide donors derived from N-acetyl lactosamine for glycosylating 3′,4′-dihydroxy disaccharide lactose acceptors with a sulphonic acid promoter has been tried. While providing a more direct reaction route, disaccharide oxazoline donors derived from N-acetyl lactosamine have had low reactivity with 3′,4′-dihydroxy disaccharide acceptors [Zurabyan et al. Soviet J. Bioorg. Chem. 1978, 4, 679]. As a result, only complex glycosylation reaction mixtures have been obtained, from which LNnT has been obtained in rather poor yields.

The use of activated 3′-monohydroxy disaccharide acceptors—having 2′, 4′ and/or 6′ electron-donating groups like benzyl, rather than electron withdrawing groups like acyls—have therefore been recommended for glycosylating with disaccharide oxazoline donors and sulphonic acid promoters [Dahmén et al. Carbohydr. Res. 1985, 138, 17; Takamura et al. Chem. Pharm. Bull. 1980, 28, 1804; 1981, 29, 2270]. This is because benzyl groups don't migrate, but they activate the near-by OH-group to be glycosylated, thereby making the disaccharide acceptor more active. However, when using an activated 3′-monohydroxy disaccharide acceptor, a large excess of a poorly reacting disaccharide oxazoline donor has still been required which, despite fair to good yields, led to unavoidable difficulties in separating the tetrasaccharide product from the large volume of unreacted oxazoline donor.

Thus, processes for chemically synthesizing LNT, LNnT and higher HMO core oligosaccharide structures in acceptable yields by glycosylating disaccharide acceptors and donors have remained complicated and expensive. There has been a need, therefore, for a simpler and more productive process which could be used for large, industrial scale production.

### BRIEF DESCRIPTION OF THE FIGURES

The invention will be described in further detail hereinafter with reference to the accompanying figures, in which:

FIG. 1 shows the ¹H-NMR spectrum of 1-O-benzyl-β-LNnH in D₂O, at 25° C. 600 MHz.

FIG. 2 shows the ¹³C-NMR spectrum of 1-O-benzyl-β-LNnH in D₂O, at 25° C. 125 MHz.

FIG. 3 shows the ¹H-NMR spectrum of 1-O-benzyl-β-para-LNnH in D₂O, at 25° C. 600 MHz.

FIG. 4 shows the ¹³C-NMR spectrum of 1-O-benzyl-β-para-LNnH in D₂O, at 25° C. 125 MHz.

### SUMMARY OF THE INVENTION

The present invention relates to a method that can be used for making an HMO core structure precursor of formula 3

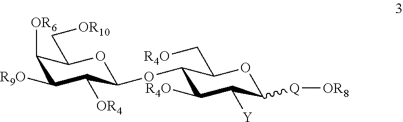

wherein R₄ is acyl, R₆ is H or acyl, preferably H, R₈ is a group removable by hydrogenolysis, Y is —OR₄ or acetylamino optionally substituted by a halogen atom, Q is a bond when Y is —OR₄ or Q is a carbohydrate linker comprising a peracylated lactose moiety optionally substituted with a peracylated N-acetyllactosaminyl residue or a peracylated lacto-N-biosyl residue when Y is an acetylamino optionally substituted by a halogen atom, R₉ is selected from the group consisting of a residue of formula B, a peracylated N-acetyllactosaminyl residue and a peracylated lacto-N-biosyl residue and R₁₀ is selected from the group consisting of a residue of formula B, acyl, acetal type groups, silyl and a peracylated N-acetyllactosaminyl residue optionally substituted with 1 or 2 peracylated N-acetyllactosaminyl moiety or lacto-N-biosyl moiety, provided that at least one of R₉ and R₁₀ is a residue of formula B

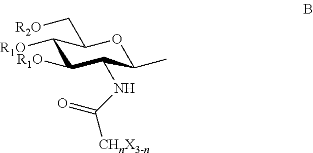

wherein X is a halogen atom selected from the group consisting of F, Cl, Br and I, n is 0, 1, 2 or 3, and one of the R₁-groups is a residue of formula A

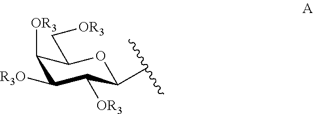

and the other R-group is acyl, and R₂ and R₃ are independently acyl,

by reacting a disaccharide glucosamine donor of formula 1

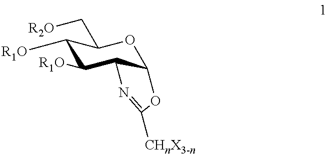

wherein R₁, R₂, X and n are as defined above,

with an acceptor derivative of formula 2

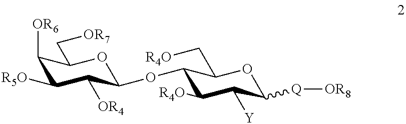

wherein R₄, R₆, R₈, Y and Q are as defined above, R₅ is selected from the group consisting of H, a peracylated N-acetyllactosaminyl residue and a peracylated lacto-N-biosyl residue, preferably H, and R₇ is selected from the group consisting of H, acyl, acetal type groups, silyl and a peracylated N-acetyllactosaminyl residue optionally substituted with 1 or 2 peracylated N-acetyllactosaminyl moiety or lacto-N-biosyl moiety, provided that at least one of R₅ and R₇ is H,
   in the presence of a boron halogenide promoter.
   In addition, it is provided compounds of formula 3′

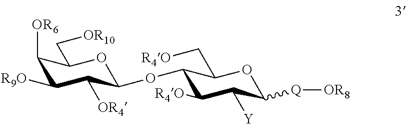

wherein R₄′ is a low-migrating acyl group, R₆ is H or acyl, preferably H, R₈ is a group removable by hydrogenolysis, Y is —OR₄′ or acetylamino optionally substituted by a halogen atom, Q is a bond when Y is —OR₄′ or Q is a carbohydrate linker comprising a peracylated lactose moiety optionally substituted with a peracylated N-acetyllactosaminyl residue or a peracylated lacto-N-biosyl residue when Y is an acetylamino optionally substituted by a halogen atom, R₉ is selected from the group consisting of a residue of formula B, a peracylated N-acetyllactosaminyl residue and a peracylated lacto-N-biosyl residue and R₁₀ is selected from the group consisting of a residue of formula B, acyl, acetal type groups, silyl and a peracylated N-acetyllactosaminyl residue optionally substituted with 1 or 2 peracylated N-acetyllactosaminyl moiety or lacto-N-biosyl moiety, provided that at least one of R₉ and R₁₀ is a residue of formula B

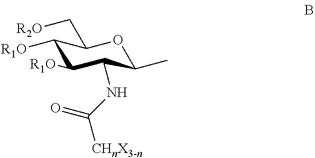

wherein X is a halogen atom selected from the group consisting of F, Cl, Br and I, n is 0, 1, 2 or 3, and one of the R₁-groups is a residue of formula A

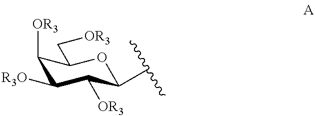

and the other R₁-group is acyl, R₂ and R₃ are independently acyl,

and compounds of formula 2B

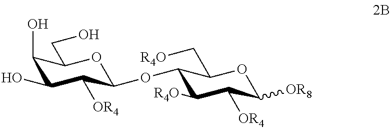

wherein R₄ is acyl and R₈ is a group removable by hydrogenolysis.

### DETAILED DESCRIPTION OF THE INVENTION

In accordance with this invention, the term “acyl” preferably means an R—C(═O)— group, wherein R can be H, linear or branched alkyl with 1-6 carbon atoms or aryl including phenyl and naphthyl, preferably phenyl, such as formyl, acetyl, propionyl, butyryl, pivaloyl, benzoyl, etc. The alkyl and aryl residues can be unsubstituted or substituted one or several times, preferably 1-5 times, more preferably 1-3 times. The substituents can preferably be alkyl (for aromatic acyl), hydroxy, alkoxy, carboxy, oxo (for alkyl, forming a keto or aldehyde function), alkoxycarbonyl, alkylcarbonyl, formyl, aryl, aryloxycarbonyl, aryloxy, arylamino, arylcarbonyl, amino, mono- and dialkylamino, carbamoyl, mono- and dialkyl-aminocarbonyl, alkylcarbonylamino, cyano, alkanoyloxy, nitro, alkylthio and/or halogen (F, Cl, Br, I). The substituents on aryl and alkyl moieties of acyl groups can modify the general chemical characteristics of the acyl group, and thereby the characteristics, such as stability, solubility and the ability to form crystals, of a molecule as a whole.

Herein, the term “group removable by hydrogenolysis” preferably means a protecting group whose C—O bond to the 1-oxygen can be cleaved by hydrogen in the presence of a catalytic amount of palladium, Raney nickel or any other conventional hydrogenolysis catalyst to regenerate a 1-OH group. Such protecting groups are described in Wuts and Greene: Protective Groups in Organic Synthesis, John Wiley & Sons, 2007, and include benzyl, diphenylmethyl (benzhydryl), 1-naphthylmethyl, 2-naphthylmethyl and triphenylmethyl (trityl) groups, each of which can be optionally substituted by one or more of the following groups: alkyl, alkoxy, phenyl, amino, acylamino, alkylamino, dialkylamino, nitro, carboxyl, alkoxycarbonyl, carbamoyl, N-alkylcarbamoyl, N,N-dialkylcarbamoyl, azido, halogenalkyl or halogen. Preferably, such substitution, if present, is on the aromatic ring(s). A preferred protecting group is benzyl optionally substituted with one or more of the following groups: phenyl, alkyl and halogen, particularly unsubstituted benzyl, 4-chlorobenzyl, 3-phenylbenzyl and 4-methylbenzyl groups. These preferred and particularly preferred protecting groups have the advantage that the by-products of their hydrogenolysis are exclusively toluene or substituted toluene. Such by-products can easily be removed, even in multi-ton quantities, from water-soluble oligosaccharide products via evaporation and/or extraction processes.

Herein, the term “peracylated N-acetyllactosaminyl moiety” in R₅, R₇ or Q preferably means the glycosyl residue of N-acetyl-lactosamine (LacNAc, Galpβ1-4GlcNAcp) linked with β-linkage and the OH-groups are protected by acyls (vide supra), preferably identical acyls:

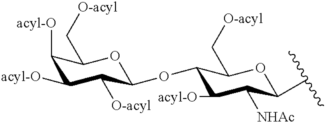

Furthermore, herein the term “peracylated lacto-N-biosyl moiety” in R₅, R₇, R₉ or Q preferably means the glycosyl residue of lacto-N-biose (LNB, Galpβ1-3GlcNAcp) linked with β-linkage and the OH-groups are protected by acyls (vide supra), preferably identical acyls:

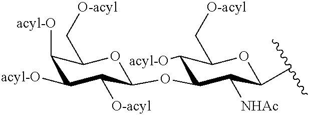

The term “peracylated N-acetyllactosaminyl residue optionally substituted by 1 or 2 moieties selected from a peracylated N-acetyllactosaminyl group and a peracylated lacto-N-biosyl group” in R₇ and R₁₀ preferably means herein that a peracylated N-acetyllactosaminyl moiety as defined above can be substituted, by replacing one or two of the acyl groups, with 1 or 2 disaccharide residues selected from the group consisting of a peracylated N-acetyllactosaminyl moiety (vide supra) and a peracylated lacto-N-biosyl moiety (vide supra).

The term “a carbohydrate linker comprising a peracylated lactose moiety optionally substituted with either a peracylated N-acetyllactosamine or a peracylated lacto-N-biose” in group Q preferably means herein a divalent group comprising a peracylated lactosyl (Galpβ1-4Glcp) residue which is linked to the OR₈ group via the anomeric carbon atom and is optionally substituted by a peracylated N-acetyllactosaminyl residue or peracylated lacto-N-biosyl residue.

Herein, the term “acyclic acetal type group” in R₇ preferably means a protective group that, with the oxygen atom of the hydroxyl group to be protected, forms a structure with two single bonded oxygens attached to the same carbon atom characterized by the following general structure:

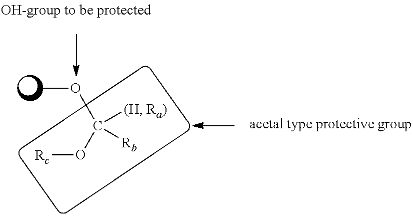

wherein Rₐ, R_{b} and R_{c} are carbon-bonded groups. This kind of groups is well-known to the person skilled in the art, many of them are referred to by P. G. M. Wuts and T. W. Greene: Protective Groups in Organic Synthesis, John Wiley & Sons, 2007, including but not limited to methoxymethyl, t-butoxymethyl, 2-methoxy-ethoxymethyl, benzyloxymethyl, tetrahydropyranyl, tetrahydrofuranyl, 1,4-dioxan-2-yl, 1-methyl-1-methoxyethyl, 1-methyl-1-phenoxyethyl, etc. The acetal type protective groups described above are labile under mild acidic conditions.

The term “silyl group” preferably means herein a protective group containing silicon atom covalently bonded to the oxygen atom of a hydroxy group to be protected (silyl ethers). This kind of groups is well-known to the person skilled in the art, many of them are referred to by P. G. M. Wuts and T. W. Greene: Protective Groups in Organic Synthesis, John Wiley & Sons, 2007, including but not limited to trimethylsilyl, triisopropylsilyl, t-butyldimethylsilyl, t-butyldiphenylsilyl, etc. The silyl ethers are labile under mild acidic conditions.

The term “a low-migrating acyl group” preferably means herein a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl. These low-migrating acyl groups are characterized by not being prone to migrate, i.e., having a low proclivity to move between —OH groups, under the reaction conditions employed.

In accordance with this invention, it has been surprisingly discovered that a disaccharide glucosamine donor of formula 1 can be coupled to a galactosyl oligosaccharide acceptor, especially a deactivated acceptor, under mild conditions to produce an unexpectedly high yield of a coupled product. This method can be used to make a wide variety of HMO core structures, even on an industrial scale.

In accordance with this invention, the method involves making a precursor of an HMO core structure by reacting a disaccharide glucosamine donor of formula 1

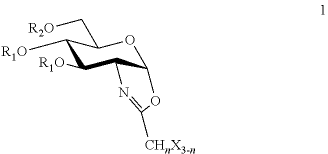

wherein X is a halogen atom selected from the group consisting of F, Cl, Br and I, n is 0, 1, 2 or 3, and one of the R₁-groups is a residue of formula A

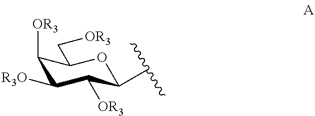

and the other R₁-group is acyl, and R₂ and R₃ are independently acyl,

with an acceptor derivative of formula 2

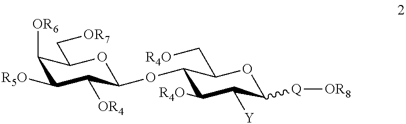

wherein R₄ is acyl, R₅ is selected from the group consisting of H, a peracylated N-acetyllactosaminyl residue and a peracylated lacto-N-biosyl residue, R₆ is H or acyl, preferably H, R₇ is selected from the group consisting of H, acyl, acetal type groups, silyl and a peracylated N-acetyllactosaminyl residue optionally substituted with 1 or 2 peracylated N-acetyllactosaminyl moiety or lacto-N-biosyl moiety, R₈ is a group removable by hydrogenolysis, Y is —OR₄ or acetylamino optionally substituted by a halogen atom, Q is a bond when Y is —OR₄ or Q is a carbohydrate linker comprising a peracylated lactose moiety optionally substituted with a peracylated N-acetyllactosaminyl residue or a peracylated lacto-N-biosyl residue when Y is an acetylamino optionally substituted by a halogen atom, provided that at least one of R₅ and R₇ is H,
   in the presence of a boron halogenide promoter. The HMO core structure obtainable by this method can be characterized by formula 3

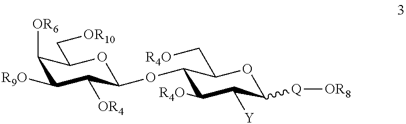

wherein R₄, R₆, R₈, Y and Q are as defined above, R₉ is selected from the group consisting of a residue of formula B, a peracylated N-acetyllactosaminyl residue and a peracylated lacto-N-biosyl residue and R₁₀ is selected from the group consisting of a residue of formula B, acyl, acetal type group, silyl and a peracylated N-acetyllactosaminyl residue optionally substituted with 1 or 2 peracylated N-acetyllactosaminyl moiety or lacto-N-biosyl moiety, provided that at least one of R₉ and R₁₀ is a residue of formula B

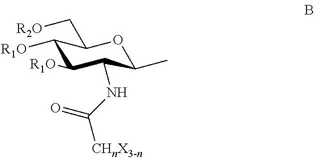

wherein R₁, R₂, X and n are as defined above.

The compounds of formula 3 include all the HMO core structures listed in Table 1, above, and their structural isomers in derivatized (protected) form.

The method of reacting the acceptor of formula 2 with the glucosaminyl donor of formula 1 can be carried out in an aprotic solvent such as acetonitrile, halogenated hydrocarbons like chloroform, dichloromethane or dichloroethane, ethers like diethyl ether, tetrahydrofuran or dioxane, aromatic hydrocarbons like toluene, benzene or xylenes, or in a mixture of aprotic solvents in the presence of a boron halogenide activator (i.e. a promoter or catalyst) so as to lead to the desired glycosylated product of formula 3. The donor-acceptor ratio varies from 1 to 3, preferably 1.2 to 1.8, more preferably 1.5 to 1.6. The amount of activator present in the glycosylation reaction is 0.1 to 0.5 equivalents relative to the donor, preferably 0.2 to 0.4, more preferably 0.25 to 0.35 equivalents. The temperature can vary from room temperature to reflux, preferably about 50-55° C. to reflux. A preferred boron halogenide activator is a boron trifluoride, more preferably boron trifluoride etherate.

In a preferred embodiment of the method, n is 3 in a compound of formula 1, thus forming a 2-methyl-oxazoline type compound. In addition, when Y is an acetylamino group optionally substituted by a halogen atom, the acetylamino is preferably non-substituted. i.e. —NHCOCH₃.

Also in a preferred embodiment of the method, when Q is an oligosaccharide linker as defined above and Y is an acetylamino group optional substituted a halogen atom, the linker can be a divalent lactosyl residue which is linked to the OR₈ group via the anomeric carbon atom, and also linked to the N-acetyllactosamine component of the derivative of formula 2 via one of the OH-groups through an interglycosidic linkage, preferably a β-linkage. The linker is peracylated preferably by identical acyl groups. More preferably, the N-acetyllactosamine component of the derivative of formula 2 can be attached to the 3′-OH of the lactosyl residue, thus the divalent linker can be shown as below:

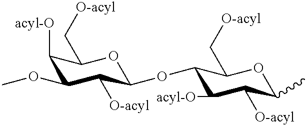

Moreover, linker Q can include a lactosyl residue substituted by an N-acetyllactosaminyl or a lacto-N-biosyl moiety, which lactosyl group can be linked to the OR₈ group via the anomeric carbon atom, and also linked to the N-acetyllactosamine component of the derivative of formula 2 via one of the OH-groups through an interglycosidic linkage, preferably a β-linkage. The substituent N-acetyllactosaminyl or lacto-N-biosyl moiety can be attached to one of the OH-groups of the lactosyl residue by a β-interglycosidic linkage, preferably to the 3′-OH group. The linker is peracylated preferably by identical acyl groups. More preferably, the N-acetyllactosamine component of the derivative of formula 2 can be attached to the 6′-OH of the lactosyl residue, thus the divalent linker can be those depicted below:

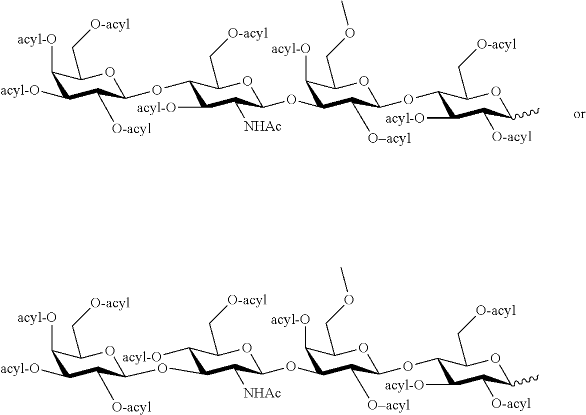

In addition, linker Q can include a lactosyl residue substituted by an N-acetyllactosaminyl moiety, which lactosyl group can be linked to the OR₈ group via the anomeric carbon atom. The substituent N-acetyllactosaminyl moiety can be attached to one of the OH-groups of the lactosyl residue by a β-interglycosidic linkage, preferably to the 3′-OH group. The linker is peracylated preferably by identical acyl groups, which can be linked to the N-acetyllactosamine component of the derivative of formula 2 via one of the OH-groups of the substituent N-acetyllactosaminyl residue through an interglycosidic linkage, preferably a β-linkage. More preferably, the N-acetyllactosamine component of the derivative of formula 2 can be attached to the 3′-OH of the substituent N-acetyllactosaminyl residue, thus the divalent linker can be as depicted below:

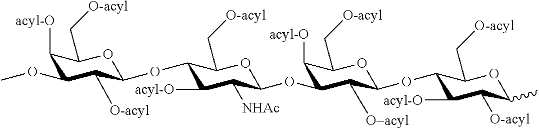

Also in a preferred embodiment of the method, the donor of formula 1 wherein n is 3 is reacted with the lactose acceptor of formula 2A

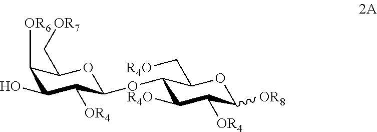

wherein R₇ is selected from the group consisting of acyl, acetal type groups and silyl, and R₄, R₆ and R₈ are as defined above,
   to produce an LNT or LNnT precursor of formula 4 belonging to compounds of formula 3

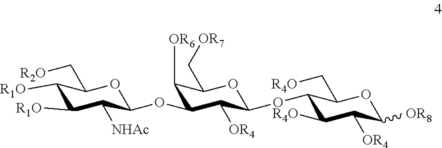

wherein R₁, R₂, R₄, R₆, R₇ and R₈ are as defined above.

In this regard, it can be particularly preferred to react an N-acetyllactosamine donor of formula 1A

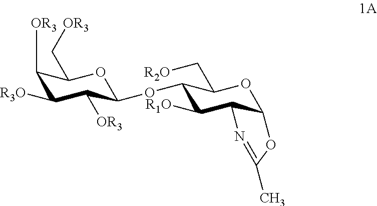

wherein R₁, R₂ and R₃ are as defined above, preferably they are identical and mean acetyl or benzoyl,
   with a lactose acceptor of formula 2A, wherein R₈ is H, R₇ is selected from the group consisting of acyl, acetal type group and silyl, preferably acyl or silyl, more preferably acyl, and R₄ and R₈ are as defined above, to produce an LNnT precursor of formula 4A

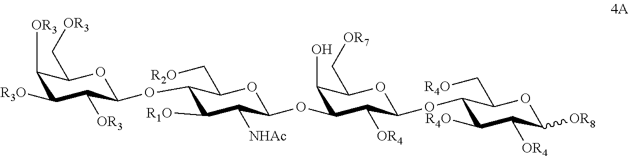

wherein R₁, R₂ and, R₃, R₄, R₇ and R₈ are as defined above,

It can be quite particularly preferred that R₄ and R₇ are identical acyl groups, particularly low-migrating acyl groups.

Also in a preferred embodiment of the method, a donor of formula 1A above can be reacted with a lactose acceptor of formula 2A, wherein R₆ is H, R₄ and R₇ are identical low-migrating acyl groups, such as a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl, R₈ is benzyl optionally substituted with one or more of the following groups: phenyl, and halogen, particularly unsubstituted benzyl, 4-chlorobenzyl, 3-phenylbenzyl and 4-methylbenzyl groups, —OR₈ is in β-orientation in a solvent selected from chloroform, dichloromethane, dichloroethane, toluene, THF and acetonitrile or mixtures thereof, especially in toluene or toluene/dichloromethane mixture, in the presence of boron trifluoride etherate promoter.

Also in a preferred embodiment of the method a lacto-N-biose donor of formula 1B

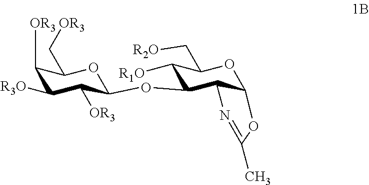

wherein R₁, R₂ and R₃ are as defined above, preferably they are identical and mean acetyl or benzoyl,
   can be reacted with a lactose acceptor of formula 2A, wherein R₆ is H, R₇ is selected from the group consisting of acyl, acetal type groups and silyl, preferably acyl or silyl, more preferably acyl, and R₄ and R₈ are as defined above, providing an LNT precursor of formula 4B

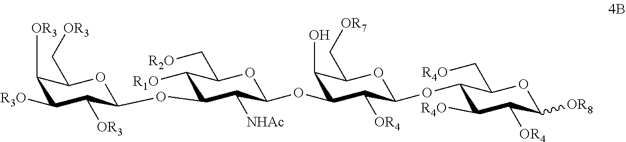

wherein R₁, R₂, R₃, R₄, R₇ and R₈ are as defined above.

In this regard, it can be particularly preferred that R₄ and R₇ are identical acyl groups, particularly low-migrating acyl groups, and it can be most particularly preferred to react, a donor of formula 1B above with a lactose acceptor of formula 2A, wherein R₈ is H, R₄ and R₇ are identical low-migrating acyl groups, such as a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl, R₈ is benzyl optionally substituted with one or more of the following groups: phenyl, alkyl and halogen, particularly unsubstituted benzyl, 4-chlorobenzyl, 3-phenylbenzyl and 4-methylbenzyl groups, and —OR₈ is in β-orientation, in a solvent selected from chloroform, dichloromethane, dichloroethane, toluene, THF and acetonitrile or a mixture thereof, especially in toluene or a toluene/dichloromethane mixture, in the presence of boron trifluoride etherate promoter.

Moreover, improved yields of an LNT precursor of formula 4B can be obtained by reacting a donor of formula 1B and a lactose acceptor of formula 2A, using at least one of the following:
molecular sieve;
1-2 equivalents extra of the donor relative to the acceptor;
additional activator(s), such as trimethylsilyl chloride (TMSCl), trimethylsilyl bromide (TMSBr), trimethylsilyl triflate (TMSOTf), triflic acid (TfOH), p-toluenesulfonic acid (pTsOH), camphorsulfonic acid (CSA), pyridinium triflate, pyridinium p-toluenesulfonate (PPTS), lanthanum triflate (La(OTf)₃), scandium triflate (Sc(OTf)₃), ytterbium triflate (Yb(OTf)₃), cupric chloride (CuCl₂) and/or cupric bromide (CuBr₂), preferably in an amount of 15-100 mol % relative to the donor.

Also in a preferred embodiment of the method, a donor of formula 1 above can be reacted with a lactose acceptor of formula 2B

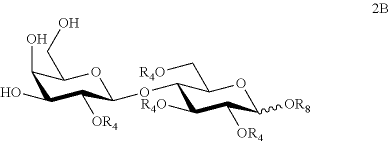

wherein R₄ is acyl and R₈ is a group removable by hydrogenolysis,

to obtain a precursor of an HMO core structure that is a hexasaccharide characterized by formula 5 belonging to compounds of formula 3

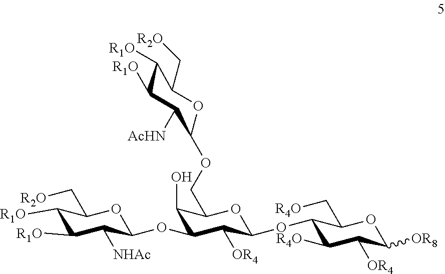

wherein R₁, R₂, R₄ and R₈ are as defined above.

In this regard, it can be particularly preferred to react an N-acetyllactosamine donor of formula 1A

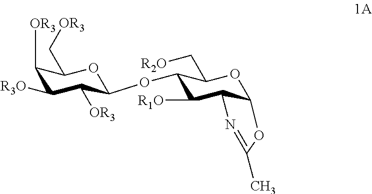

wherein R₁, R₂ and R₃ are as defined above, preferably they are identical and mean acetyl or benzoyl,
   with a lactose acceptor of formula 2B above to produce an LNnH (lacto-N-neohexaose) precursor of formula 5A

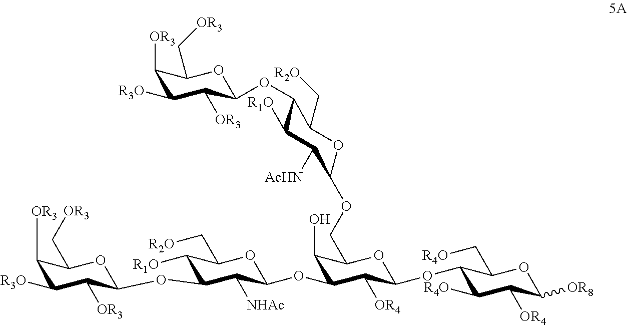

wherein R₁, R₂, R₃, R₄ and R₈ are as defined above.

It can be quite particularly preferred that is a low-migrating acyl group, and it can be most particularly preferred to react a donor of formula 1A wherein R₁, R₂ and R₃ are identical and are acetyl or benzoyl with a lactose acceptor of formula 2B, wherein R₄ is a low-migrating acyl group, such as a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl, R₈ is benzyl optionally substituted with one or more of the following groups: phenyl, alkyl and halogen, particularly unsubstituted benzyl, 4-chlorobenzyl, 3-phenylbenzyl and 4-methylbenzyl groups, and —OR₈ is in β-orientation, in a solvent selected from chloroform, dichloromethane, dichloroethane, toluene, THF and acetonitrile or a mixture thereof, especially in toluene or a toluene/THF mixture, in the presence of boron trifluoride etherate promoter.

Also in a preferred embodiment of the method, a donor of formula 1A

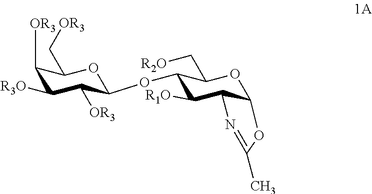

wherein R₁, R₂ and R₃ are as defined above, preferably they are identical and mean acetyl or benzoyl,
   can be reacted with an acceptor of formula 2C or 2D

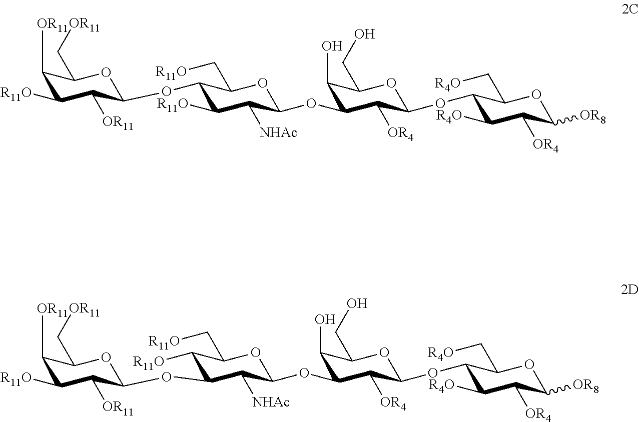

wherein R₄ and R₁₁ are independently acyl, and R₈ is a group removable by hydrogenolysis,
   to produce a precursor of an HMO core structure that is an LNnH (lacto-N-neohexaose) precursor of formula 5B or an LNH (lacto-N-hexaose) precursor of formula 5C, respectively, both belonging to compounds of formula 3

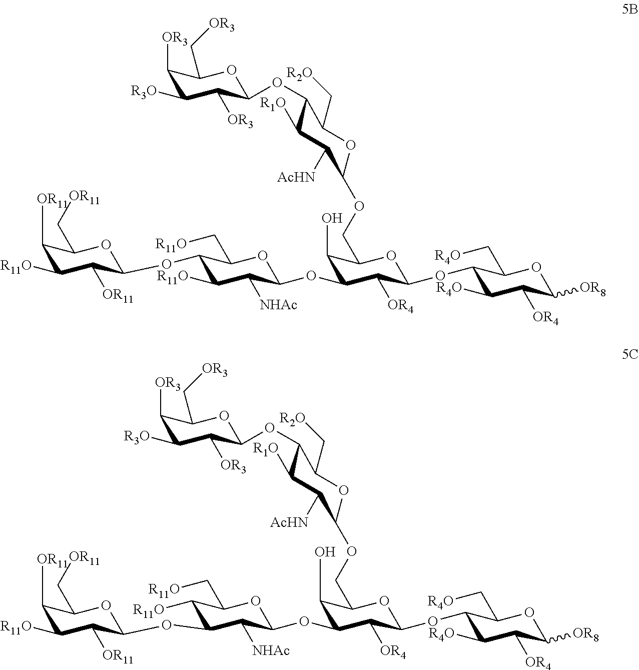

wherein R₁, R₂, R₃, R₄, R₈ and R₁₁ are as defined above.

In this regard, it can be particularly preferred that R₄ is a low-migrating acyl group, and it can be most particularly preferred to react a donor of formula 1A wherein R₁, R₂ and R₃ are identical and are acetyl or benzoyl, with a lactose acceptor of formula 2C or 2D, wherein R₄ is a low-migrating acyl group, such as a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl, R₈ is benzyl optionally substituted with one or more of the following groups: phenyl, alkyl and halogen, particularly unsubstituted benzyl, 4-chlorobenzyl, 3-phenylbenzyl and 4-methylbenzyl groups, —OR₈ is in β-orientation, and R₁₁ is acetyl or benzoyl, preferably acetyl, in a solvent selected from chloroform, dichloromethane, dichloroethane, toluene, THF and acetonitrile or a mixture thereof, especially in toluene or a toluene/dichloromethane mixture, in the presence of boron trifluoride etherate promoter.

Also in a preferred embodiment of the method, a donor of formula 1 above can be reacted with a tetrasaccharide acceptor of formula 2E

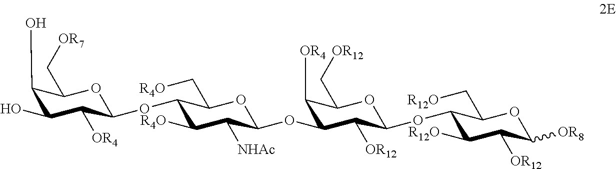

wherein R₄ is acyl, R₇ is selected from the group consisting of acyl, acetal type group

and silyl, preferably acyl, R₈ is a group removable by hydrogenolysis, and R₁₂ is acyl, to produce a precursor of an HMO core structure that is a linear hexasaccharide characterized by formula 6 belonging to compounds of formula 3

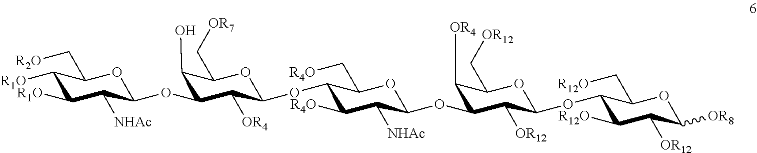

wherein R₁, R₂, R₄, R₇, R₈ and R₁₂ are as defined above.

In this regard, it can be particularly preferred that an N-acetylglucosamine donor of formula 1A or 1B

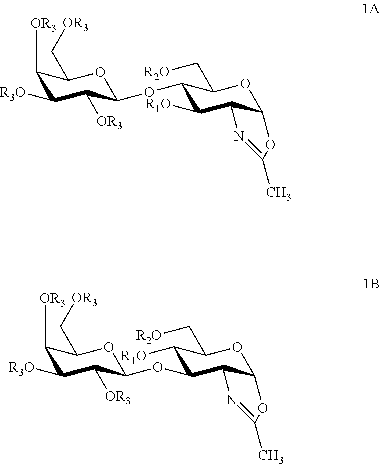

wherein R₁, R₂ and R₃ are as defined above, preferably they are identical and mean acetyl or benzoyl,
   is reacted with a tetrasaccharide acceptor of formula 2E above to produce a para-LNnH (para-lacto-N-neohexaose) precursor of formula 6A or a para-LNH (para-lacto-N-hexaose) precursor of formula 6B, respectively

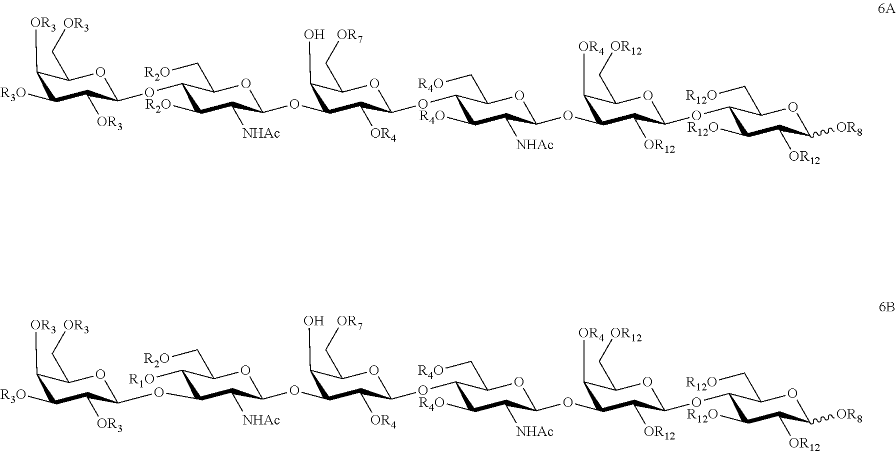

wherein R₁, R₂, R₃, R₄ R₇, R₈ and R₁₂ are as defined above.

It can be quite particularly preferred that R₄ is a low-migrating acyl group, and it can be most particularly preferred to react a donor of formula 1A wherein R₁, R₂ and R₃ are identical and are acetyl or benzoyl, with a lactose acceptor of formula 2E, wherein R₄ and R₇ are identical and are acyl, particularly low-migrating acyl group, such as a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl, R₈ is benzyl optionally substituted with one or more of the following groups: phenyl, alkyl and halogen, particularly unsubstituted benzyl, 4-chlorobenzyl, 3-phenylbenzyl and 4-methylbenzyl groups, —OR₈ is in β-orientation, and R₁₁ is acyl, particularly low-migrating acyl group, such as a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl, in a solvent selected from chloroform, dichloromethane, dichloroethane, toluene, THF and acetonitrile or a mixture thereof, especially in toluene or toluene/dichloromethane mixture, in the presence of boron trifluoride etherate promoter.

Compounds of formula 1 can be prepared by known methods, for example: i) from the corresponding N-acetyl peracyl derivatives by treating with trimethylsilyl trifluoromethanesulphonate [e.g. Range et al. Tetrahedron 1997, 53 1695, Sato et al. J. Carbohydr. Chem. 1998, 17, 703] or ii) from the corresponding N-acetyl peracyl derivatives by treating with hydrazine then mesyl chloride [e.g. Bovin et al. Bull. Acad. Sci. USSR, Div. Chem. Sci. 1981, 30, 2339], or iii) from the corresponding glycosyl chloride by treating with silver triflate [e.g. Kaji et al. Heterocycles 2004, 64, 317]. Preferred compounds according to formula 1 used in the method of this invention as a donor are those having the R₁-group not being the residue A, R₂ and R₃ are identical and are acetyl or benzoyl.

Compounds of formula 2A, wherein R₄ and R₇ are identical, and R₆ means H or acyl, can be prepared, via Scheme 1, below, by first treating octa-O-acetyl lactose or hepta-O-acetyl lactosyl bromide with an alcohol of formula R₈OH under activation conditions for a Lewis acid (e.g. a mercury salt, BFa-etherate). After de-O-acetylation (e.g. Zemplén-deprotection, aminolysis or basic hydrolysis) followed by regioselective acetonidation with dimethoxypropane in the presence of an acid catalyst results in a 3′,4′-protected lactoside that can then be acylated with R₄-halogenide or (R₄)₂O (anhydride) under conventional conditions. The resulting derivative can be hydrolysed with acid to remove the protective isopropylidene group to give a diol of formula 2, wherein R₅ and R₆ are H, which can optionally be treated with an orthoester. A cyclic orthoester thus obtained can subsequently be rearranged with an acid catalyst to form another compound of formula 2, wherein R₆ is acyl [see e.g. Paulsen et al. Carbohydr. Res. 1985, 137, 39; Lubineau et al. ibid. 1997, 305, 501; and references cited therein]. In preferred compounds according to formula 2A used in the method as acceptor R₆ is H, R₈ is benzyl optionally substituted with one or more of the following groups: phenyl, alkyl and halogen, particularly unsubstituted benzyl, 4-chlorobenzyl, 3-phenylbenzyl and 4-methylbenzyl groups, —OR₆ is in β-orientation, and R₄ acyl groups are low-migrating acyl groups, such as a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl. These low-migrating acyl groups are characterized by not being prone to migrate, i.e., having a low proclivity to move between —OH groups, under acidic conditions.

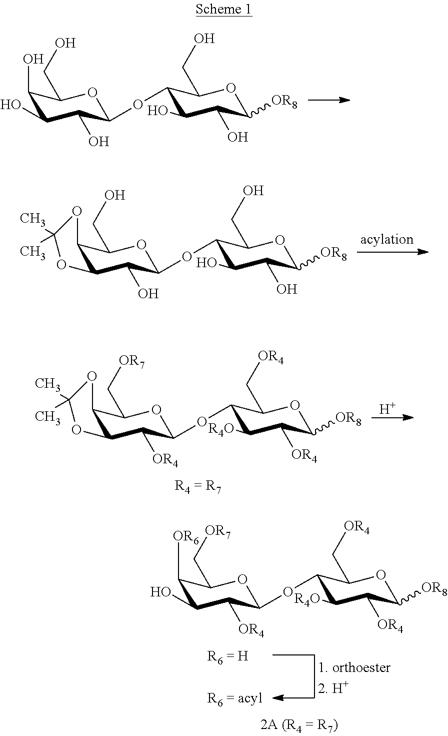

A lactose acceptor of formula 2A, wherein R₆ is H, and R₇ is an acetal protecting group or silyl, can be prepared from the triol compound of formula 2B (vide infra) by selective protection of the primary OH. Selective acyclic acetal formation of the resulting diol on the 6′-position can be performed with for example methoxymethyl, t-butoxymethyl, 2-methoxyethyl, benzyloxymethyl, 2-tetrahydrofuranyl halogenides, etc. in the presence of triethylamine, morpholine, diisopropyl ethylamine, pyridine, etc., or with for example dihydropyran, 1,4-dihydrodioxin, dihydrofuran, 2-methoxypropene, 2-phenoxypropene, etc. in the presence of acids in organic solvents such as DMF, THF, dioxane, acetonitrile, etc. at 0-60° C. temperature to give rise to compounds of formula 2A wherein R₇ is an acetal type group. Selective primary OH-silylation reaction of the lactoside diol with a silyl chloride in the presence of an amine base (such as imidazole, triethyl amine, etc.) at room temperature or with a silyl triflate with a hindered amine base (e.g. 2,6-lutidine) at low temperature can lead to a compound of general formula 2A wherein R₇ is silyl.

A lactose triol acceptor of formula 2B can be prepared in the following way (see Scheme 2). The R₈O-lactoside (vide supra) is protected selectively with benzaldehyde, substituted benzaldehyde like 4-methoxy-benzaldehyde, benzophenone or di-O-acetals thereof in a known manner giving rise to a 4′,6′-protected derivative, which is then acylated by acyl halogenide or anhydride under conventional conditions. The benzylidene acetal is removed by acidic treatment and the primary OH is selectively silylated. The acyl groups are then removed in a base catalysed transesterification reaction or a basic hydrolysis reaction. Regioselective acetonidation with dimethoxypropane in the presence of an acid catalyst gives to 3′,4′-di-O-isopropylidene-6-O-silyl lactoside derivative which is then acylated with R₄-halogenide or (R₄)₂O (anhydride) under conventional conditions. The isopropylidene and silyl protecting group are removed by acid treatment resulting in a compound of formula 2B. Alternatively, R₈O-lactoside can be regioselectively allylated via the 3′,4′-stannylidene acetal by adding allyl halogenide. Benzylidenation followed by acylation and acidic hydrolysis are then carried out as described above to get the 3′-allylated diol intermediate, from which either a compound of formula 2B can be obtained after removing the allyl group selectively through isomerization of the olefin to vinyl ether and subsequent cleavage, or a compound of formula 2A, wherein R₇ is silyl or acyclic acetal, can be achieved after silylation/acyclic acetalization and deallylation.

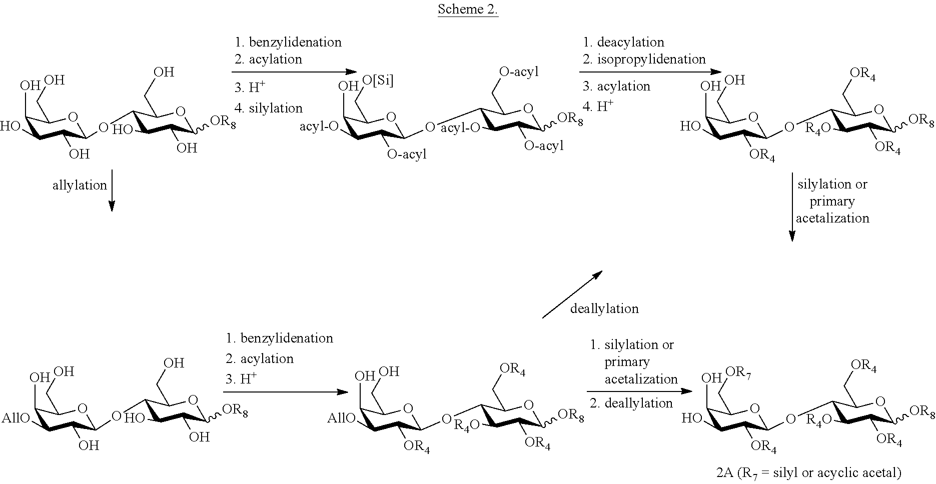

The tetrasaccharide product of formula 4 above can be transformed in a few steps into an acceptor suitable for further glycosylation reaction. As an example, the conversion of a compound of formula 4A to a compound of formula 2E is illustrated herein (see Scheme 3). A compound of formula 4A in which R₁, R₂ and R₃ are acetyl, and R₄ and R₁ are identical and are acyl except for acetyl, preferably a low-migrating acyl, and obtained in a glycosylation reaction disclosed above, is treated with acid to remove acetyls in a selective manner while the other acyls not being acetyl (R₄ and R₇) are not affected. Regioselective isopropylidenation of the so deprotected terminal galactosyl residue blocks the 3- and 4-OH groups, and the remaining OHs are acylated. Removing the cyclic acetal protection under acidic condition readily provides a compound of formula 2E suitable for glycosylation at position 4. The method set forth above is generally applicable to transform any product of formula 3 having similar protection pattern to an acceptor to be elongated at position 4 of the terminal galactosyl residue.

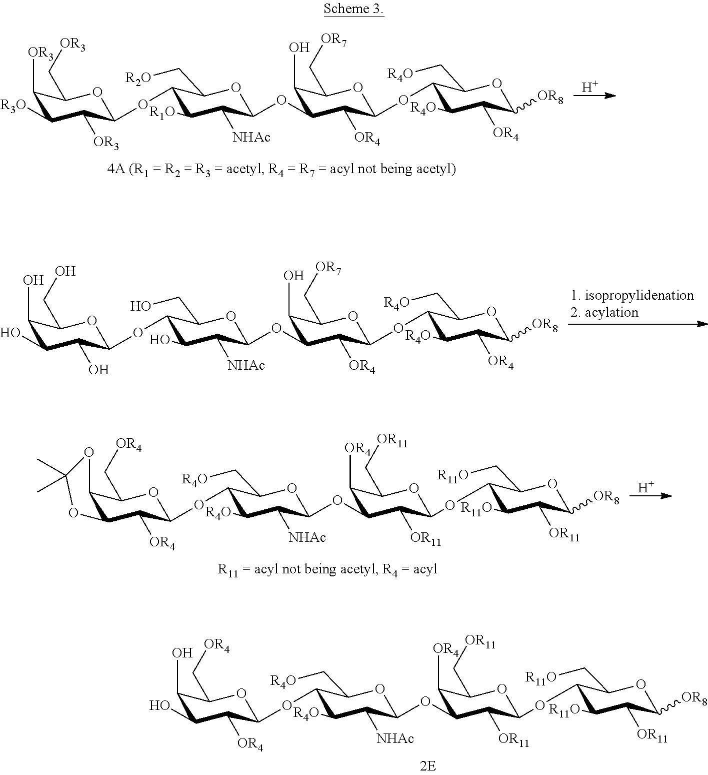

A precursor of an HMO core structure obtainable by the method of this invention can have a similar protection pattern to that of a compound of formula 4A in Scheme 3 above and can be deprotected by selective acidic deacetylation. Performing the transformation steps as shown in Scheme 2 above can lead to a triol acceptor of formula 2 wherein R₅, R₆ and R₇ are H.

Moreover, a precursor of an HMO core structure of formula 3, wherein R₁₀ is an acyclic acetal groups or silyl, obtainable by the method, can be treated with acid to remove these acid-sensitive protective group and to free the affected primary OH-group for further glycosylation according to the method of this invention. Alternatively, the silyl group can be removed with fluoride. As an example, a compound of formula 4A wherein R₁ is silyl or acyclic acetal can be de-O-silylated/de-O-acetalated to a compound of formula 2C:

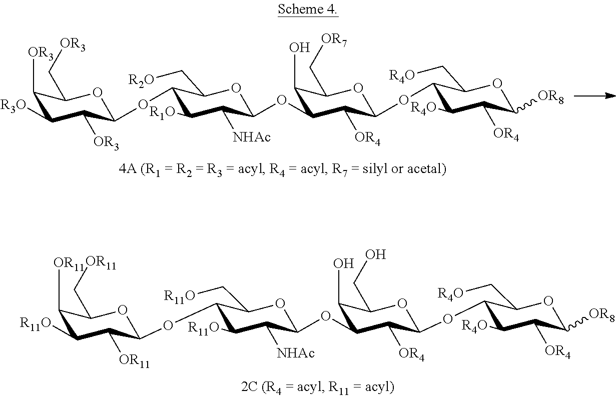

The compounds of formula 3 obtainable by the method of this invention can be useful intermediates for making HMO core structures such as those listed in Table 1 above, as only two or three types, preferably two types of protective groups are needed to be removed.

Accordingly, an acyclic acetal groups or a silyl group in R₁₀ of a compound of formula 3 can be split under acidic conditions. The starting compound may contain acyl protective groups as well. The skilled person is fully aware that acyl groups can be deprotected by only extremely strong acidic hydrolysis (pH<1). The skilled person is able to distinguish which deprotective condition affects the acetal/silyl group while the acyl groups remain intact. Furthermore, the interglycosidic linkages and the aglycon of the glucosyl residue may be also sensitive to acids. The skilled person is fully aware that interglycosidic linkages and anomeric protecting groups can be split by only strong acidic hydrolysis (pH<1-2). The skilled person is able to distinguish which deprotective condition affects the acetal/silyl group while the interglycosidic linkages remain intact. Water—which has to be present in the reaction milieu as reagent—can serve as solvent or co-solvent as well. Organic protic or aprotic solvents which are stable under acidic conditions and miscible fully or partially with water or with the aqueous solution of the acid such as C₁-C₆ alcohols, acetone, THF, dioxane, ethyl acetate, MeCN, dichloromethane, etc. can be used in a mixture with water. The acids used are generally protic acids selected from but not limited to acetic acid, trifluoroacetic acid, HCl, formic acid, sulphuric acid, perchloric acid, oxalic acid, p-toluenesulfonic acid, benzenesulfonic acid, cation exchange resins, etc., which can be present in from catalytic amount to large excess. The hydrolysis can be conducted at temperatures between 20° C. and reflux until reaching completion which takes from about 2 hours to 3 days depending on temperature, concentration and pH. Preferably, organic acids including but not limited to aqueous solutions of acetic acid, formic acid, chloroacetic acid, oxalic acid, etc. are used. Another preferred condition is to use a C₁-C₆ alcohol-acetonitrile or C₁-C₆ alcohol-water mixture in the presence of HCl or sulfonic acids such as p-toluenesulfonic acid or champhorsulfonic acid. Alternatively, anhydrous C₁-C₆ alcohol including but not limited to methanol, ethanol, propanol, butanol, etc. can also be used for the required cleavage of the acyclic acetal type moieties via acid catalysed trans-acetalization processes. Catalytic amount of hydrogen chloride, sulphuric acid, perchloric acid, p-toluenesulfonic acid, acetic acid, oxalic acid, champhorsulfonic acid, strong acidic ion-exchange resins, etc. can be used at temperatures of 20° C. to reflux. Silyl ether can be readily cleaved with HF-based reagents like e.g. Bu₄NF, KF, HF.pyridine complex, aqueous HF, NH₄F, CsF, H₂SiF₆.

In a base catalysed transesterification reaction, O-acyl protective groups for hydroxyl groups are removed in an alcohol solvent such as methanol, ethanol, propanol, t-butanol, etc. in the presence of an alcoholate, such as, NaOMe, NaOEt or KO^{t}Bu, at a temperature of 20-100° C. The alcohol solvent and the alcoholate are preferably matched, that is to say, for example, that an ethanol solvent should be used with NaOEt. Furthermore, the use of a co-solvent such as toluene or xylene can be beneficial to control particle size of the product and to avoid gel formations. Preferably, a catalytic amount of NaOMe is used in methanol (Zemplén de-O-acylation).

In a basic hydrolysis reaction, O-acyl protective groups for hydroxyl groups are removed by a base catalysed hydrolysis in water, alcohol or water-organic solvent mixtures, in homogeneous or heterogeneous reaction conditions at temperatures of 0-100° C. The base of choice is generally a strong base, such as LiOH, NaOH, KOH, Ba(OH)₂, K₂CO₃, basic ion exchange resins, tetraalkylammonium hydroxides, etc. The base can be used in an aqueous solution. Thereby, only O-acyls are removed, and the acetamido group is not affected. Preferably, the base is NaOH, and the solvent is methanol.

Thus a preferred embodiment of the method can include a further step of de-O-acylation and optional de-O-silylation/de-O-acetalization of a compound of general formula 3 to obtain a R₈-glycoside of an HMO core structure characterized by formula 7

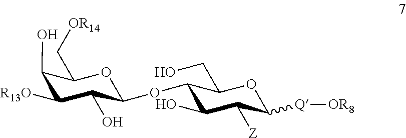

wherein R₈ is a group removable by hydrogenolysis, Z is —OH or acetylamino optionally substituted by a halogen atom, Q′ is a bond when Y is —OH or Q′ is a carbohydrate linker comprising a lactose moiety optionally substituted with an N-acetyllactosaminyl residue or a lacto-N-biosyl residue when Z is an acetylamino optionally substituted by a halogen atom, R₁₃ is selected from the group consisting of a residue of formula C, N-acetyllactosaminyl residue and lacto-N-biosyl residue, R₁₄ is selected from the group consisting of H, a residue of formula C, and N-acetyllactosaminyl residue optionally substituted with 1 or 2 N-acetyllactosaminyl moiety or lacto-N-biosyl moiety, provided that at least one of R₁₃ and R₁₄ is a residue of formula C

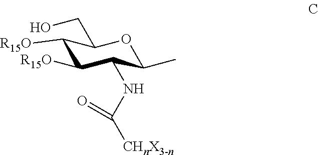

wherein one of the R₁₅-groups is a 3-D-galactopyranosyl group and the other R₁₅-group is H.

In this regard, it can be especially preferred that n is 3 in the residue of formula C and when Z is an acetylamino group optionally substituted with a halogen atom, the acetylamino group is preferably —NHCOCH₃.

In another preferred embodiment of the method of the invention, when Q′ is an oligosaccharide linker as defined above in a compound of formula 7 and Z is an acetylamino group optional substituted with a halogen atom, the linker can be a divalent lactosyl residue which is linked to the OR₈ group via the anomeric carbon atom, and also linked to the N-acetyllactosamine component of the derivative of formula 7 via one of the OH-groups through an interglycosidic linkage, preferably a β-linkage. More preferably, the N-acetyllactosamine component of the derivative of formula 7 is attached to the 3′-OH of the lactosyl residue, and thus the divalent linker can be as depicted below:

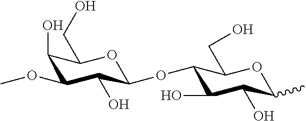

Moreover, the linker Q′ can include a lactosyl residue substituted by an N-acetyllactosaminyl or a lacto-N-biosyl moiety, which lactosyl group is linked to the OR₈ group via the anomeric carbon atom, and also linked to the N-acetyllactosamine component of the derivative of formula 7 via one of the OH-groups through an interglycosidic linkage, preferably a β-linkage. The substituent N-acetyllactosaminyl or lacto-N-biosyl moiety is attached to one of the OH-groups of the lactosyl residue by a β-interglycosidic linkage, preferably to the 3′-OH group. More preferably, the N-acetyllactosamine component of the derivative of formula 7 is attached to the 6′-OH of the lactosyl residue, thus the divalent linker can be those depicted below:

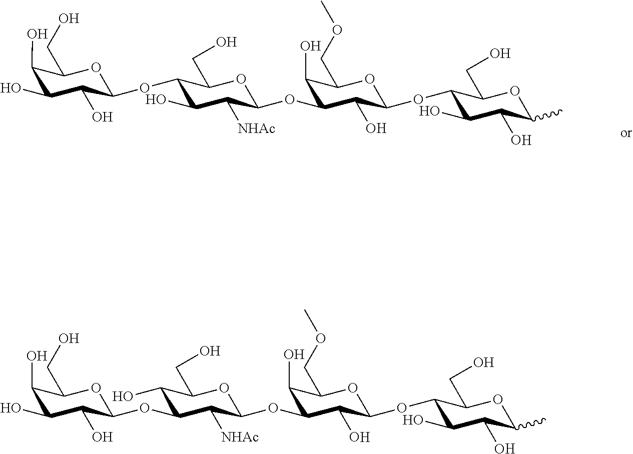

In addition, linker Q′ can include a lactosyl residue substituted by an N-acetyllactosaminyl moiety, which lactosyl group is linked to the OR₈ group via the anomeric carbon atom. The substituent N-acetyllactosaminyl moiety is attached to one of the OH-groups of the lactosyl residue by a β-interglycosidic linkage, preferably to the 3′-OH group. The linker is attached to the N-acetyllactosamine component of the derivative of formula 7 via one of the OH-groups of the substituent N-acetyllactosaminyl residue through an interglycosidic linkage, preferably a β-linkage. More preferably, the N-acetyllactosamine component of the derivative of formula 7 is attached to the 3′-OH of the substituent N-acetyllactosaminyl residue, thus the divalent linker can be as depicted below:

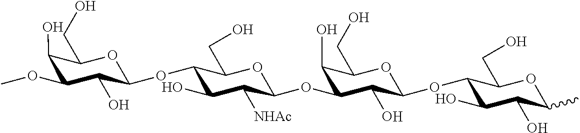

In a more preferred embodiment, the R₈-glycoside of an HMO core structure of formula 7 is a R₈-glycoside of LNT or LNnT characterized by formula 8

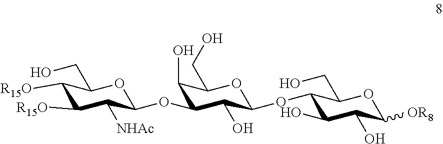

wherein R₈ and R₁₅ are as defined above,

which can be made from a compound of formula 4 above, preferably from a compound of formula 4 wherein R₇ is acyl, particularly wherein R₄ is a low-migrating acyl group, by a de-O-acylation reaction, preferably a base catalysed transesterification reaction or a basic hydrolysis reaction.

Also more preferably, the R-glycoside of an HMO core structure of formula 7 is a R₈-glycoside of LNnH, which can be made from a compound of formula 5A or 5B above, preferably from a compound of formula 5A or 5B wherein R₄ is a low-migrating acyl group, by a de-O-acylation reaction, preferably a base catalysed transesterification reaction or a basic hydrolysis reaction.

Also more preferably, the R-glycoside of an HMO core structure of formula 7 is a R-glycoside of para-LNnH, which can be made from a compound of formula 6A above, preferably from a compound of formula 6A wherein R₇ is acyl, particularly wherein R₄ is a low-migrating acyl group, by a de-O-acylation reaction, preferably a base catalysed transesterification reaction or a basic hydrolysis reaction.

Removal of the R-aglycon and thus restoring the anomeric OH-group typically takes place in a protic solvent or in a mixture of protic solvents. The protic solvent can be selected from the group consisting of water, acetic acid and C₁-C₆ alcohols. A mixture of one or more protic solvents with one or more appropriate aprotic organic solvents miscible partially or fully with the protic solvent(s), such as THF, dioxane, ethyl acetate or acetone, can also be used. Water, one or more C₁-C₆ alcohols or a mixture of water and one or more C₁-C₆ alcohols are preferably used as the solvent system. Solutions or suspensions containing the compounds to be hydrogenolysed in any concentration can be used. The reaction mixture is stirred at a temperature of from 10 to 100° C., preferably from 20 to 60° C., in a hydrogen atmosphere of from 1 to 50 bar in the presence of a catalyst such as palladium, Raney nickel or any other appropriate metal catalyst, preferably palladium on charcoal or palladium black, until completion of the reaction. Catalyst concentrations generally range from 0.1% to 10% (based on the weight of the compound of formula 7). Preferably, the catalyst concentrations range from 0.15% to 5%, more preferably 0.25% to 2.25%. Transfer hydrogenolysis can also be carried out, wherein the hydrogen is generated in situ from cyclohexene, cyclohexadiene, formic acid or ammonium formate. Addition of organic or inorganic bases/acids and/or basic and/or acidic ion exchange resins can also be used to improve the kinetics of the catalytic hydrogenolysis. The use of basic substances is especially preferred when halogen substituents are present on the substituted benzyl moieties of the precursors. Preferred organic bases include triethylamine, diisopropyl ethylamine, ammonia, ammonium carbamate, or diethylamine. Preferred organic/inorganic acids include formic acid, acetic acid, propionic acid, chloroacetic acid, dichloroacetic acid, trifluoroacetic acid, HCl, or HBr. These conditions allow for the simple, convenient and delicate removal of the anomeric protecting group to yield a pure HMO core structure which can be isolated from the reaction mixture using conventional work-up procedures in crystalline, amorphous solid, syrupy form or in a concentrated aqueous solution. It should be emphasized, that a functional group wherein n is 0, 1 or 2, and/or a Z-group meaning an acetylamino group substituted a halogen atom in a compound of formula 7 is readily transformed to acetylamino under the conditions employed.

In a preferred realization, R₈ is benzyl optionally substituted with one or more of the following groups: phenyl, alkyl and halogen, particularly unsubstituted benzyl, 4-chlorobenzyl, 3-phenylbenzyl and 4-methylbenzyl groups, and —OR₈ is in β-orientation. More preferably, 1-O-benzyl LNT, 1-O-benzyl LNnT, 1-O-benzyl LNnH or 1-O-benzyl para-LNnH is subjected to catalytic hydrogenolysis to give LNT, LNnT, LNnH or para-LNnH, respectively. The hydrogenation can be performed in water or aqueous alcohol, preferably in a water/methanol or water/ethanol mixture (alcohol content: 10-50 v/v %) at 15-65° C., preferably 60-65° C. The concentration of the 1-O-benzyl derivatives can be 140-230 g/l, and the catalyst concentration can be from 0.4% to 1.2% (weight of the metal content based on the weight of the 1-O-benzyl derivatives) [see WO 2011/100980].

The compounds of formula 3 are valuable intermediates for making HMO core structures like LNT, LNnT, LNnH or para-LNnH, as well as for producing oligosaccharides having LNT or LNnT motif such as Lewis^{a} or Lewis^{x} type compounds. Particularly valuable are the compounds of formula 3′ of this invention

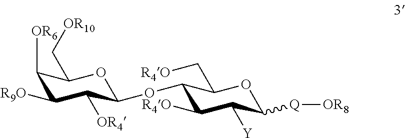

wherein R₄′ is a low-migrating acyl group, R₆ is H or acyl, preferably H, R₈ is a group removable by hydrogenolysis, Y is —OR₄′ or acetylamino optionally substituted by a halogen atom, Q is a bond when Y is —OR₄′ or Q is a carbohydrate linker comprising a peracylated lactose moiety optionally substituted with a peracylated N-acetyllactosaminyl residue or a peracylated lacto-N-biosyl residue when Y is an acetylamino optionally substituted by a halogen atom, R₉ is selected from the group consisting of a residue of formula B, a peracylated N-acetyllactosaminyl residue and a peracylated lacto-N-biosyl residue and R₁₀ is selected from the group consisting of a residue of formula B, acyl, acetal type groups, silyl and a peracylated N-acetyllactosaminyl residue optionally substituted with 1 or 2 peracylated N-acetyllactosaminyl moiety or lacto-N-biosyl moiety, provided that at least one of R₉ and R₁₀ is a residue of formula B

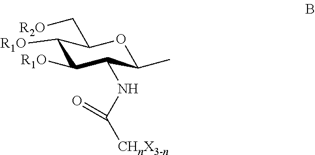

wherein X is a halogen atom selected from the group consisting of F, Cl, Br and I, n is 0, 1, 2 or 3, and one of the R₁-groups is a residue of formula A

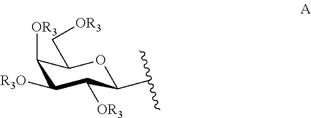

and the other R₁-group is acyl, and R₂ and R₃ are independently acyl.

A compound of formula 3′ can be either an α- or β-anomer or an anomeric mixture of α- and β-anomers. The HMO core structure precursors of formula 3′ can be crystalline solids, oils, syrups, precipitated amorphous material or spray dried products. If crystalline, a compound of formula 3′ could exist either in an anhydrous or hydrated crystalline form by incorporating one or several molecules of water into its crystal structure. Likewise, a compound of formula 3′ could exist as a crystalline substance, incorporating ligands such as organic molecules and/or ions into its crystal structure.

In a preferred compound of formula 3′, n is 3 and, when Y is an acetylamino group optionally substituted a halogen atom, the acetylamino is non-substituted meaning —NHCOCH₃.

In another preferred compound of formula 3′, is provided an LNT or LNnT precursor of formula 4′

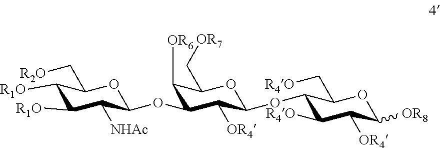

wherein R₁, R₂, R₄′, R₆ and R₈ are as defined above, and R₇ is selected from the group consisting of acyl, acetal type group and silyl, preferably acyl or silyl, more preferably acyl.

Particularly preferred compound is an LNnT precursor of formula 4A′

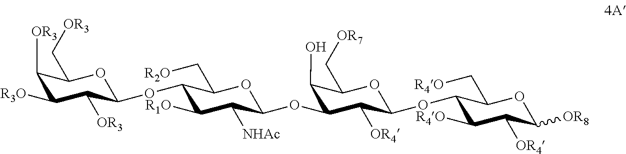

wherein R₁, R₂ and R₃ are as defined above and preferably are identical and are acetyl or benzoyl, R₇ is selected from the group consisting of acyl, acetal type groups and silyl, preferably acyl or silyl, more preferably acyl, and R₄′ and R₈ are as defined above. More preferably R₄′ and R₇ are identical low-migrating acyl groups being a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl, R₈ is benzyl optionally substituted with one or more of the following groups: phenyl, alkyl and halogen, particularly unsubstituted benzyl, 4-chlorobenzyl, 3-phenylbenzyl and 4-methylbenzyl groups, and —OR₈ is in β-orientation.

Also particularly preferred compound is an LNT precursor of formula 4B′

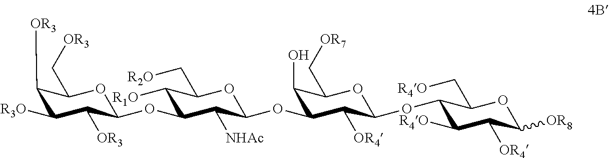

wherein R₁, R₂ and R₃ are as defined above and preferably are identical and are acetyl or benzoyl, R₇ is selected from the group consisting of acyl, acetal type group and silyl, preferably acyl or silyl, more preferably acyl, and R₄′ and R₈ are as defined above. More preferably R₄′ and R₇ are identical low-migrating acyl groups being a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl, R₈ is benzyl optionally substituted with one or more of the following groups: phenyl, alkyl and halogen, particularly unsubstituted benzyl, 4-chlorobenzyl, 3-phenylbenzyl and 4-methylbenzyl groups, and —OR₈ is in β-orientation.

In another preferred compound of formula 3′ is provided a precursor of a hexasaccharide HMO core structure of formula 5′ belonging to compounds of formula 3′

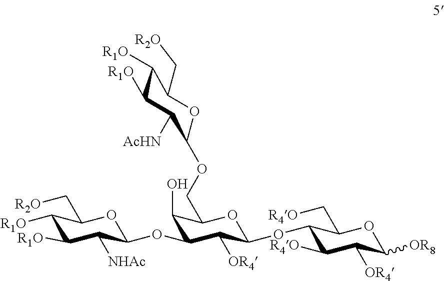

wherein R₁, R₂, R₄′ and R₈ are as defined above,

particularly an LNnH (lacto-N-neohexaose) precursor of formula 5A′

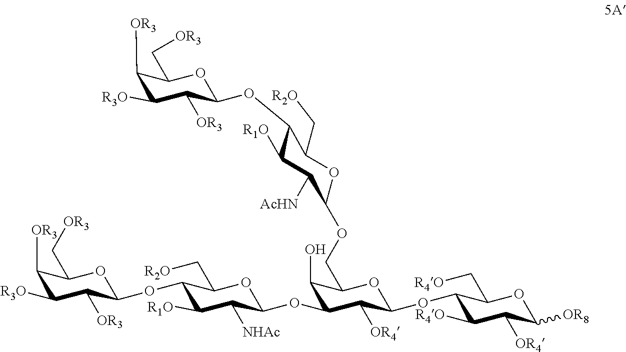

wherein R₁, R₂ and R₃ are as defined above and preferably are identical and are acetyl or benzoyl, and R₄′ and R₈ are as defined above,
   or an LNnH precursor of formula 5B′

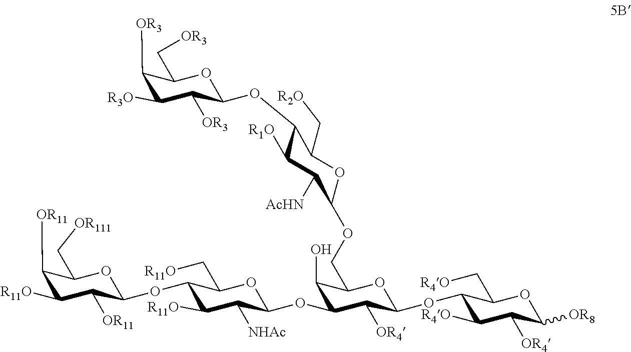

wherein R₁, R₂ and R₃ are as defined above and preferably are identical and are acetyl or benzoyl, R₄′ and R₈ are as defined above, and R₁₁ is acyl, preferably acetyl or benzoyl.

More preferably in a compound of 5A′ or 5B′ R₄′ is a low-migrating acyl group being a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl, R₈ is benzyl optionally substituted with one or more of the following groups: phenyl, alkyl and halogen, particularly unsubstituted benzyl, 4-chlorobenzyl, 3-phenylbenzyl and 4-methylbenzyl groups, and —OR₈ is in β-orientation.

Also a preferred compound of formula 3′ is the linear hexasaccharide precursor of formula 6′

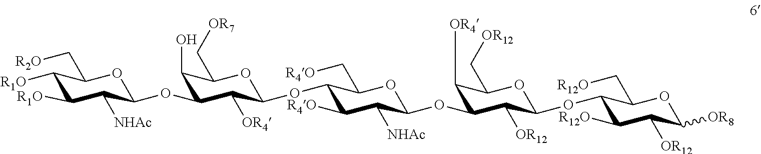

wherein R₁, R₂, R₄′ and R₈ are as defined above, R₇ is selected from the group

consisting of acyl, acetal type groups and silyl, preferably acyl, and R₁₂ is acyl, particularly a para-LNnH precursor of formula 6A′

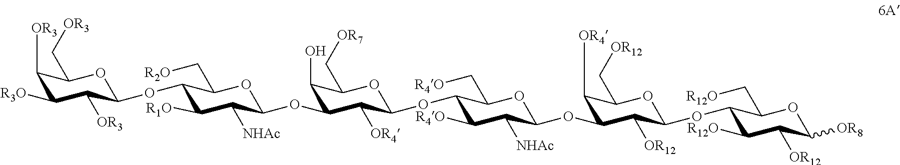

wherein R₁, R₂ and R₃ are as defined above and preferably are identical and are acetyl or benzoyl, and R₄′, R₇, R₈ and R₁₂ are as defined above.

More preferably R₄′ is a low-migrating acyl group being a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl, R₈ is benzyl optionally substituted with one or more of the following groups: phenyl, alkyl and halogen, particularly unsubstituted benzyl, 4-chlorobenzyl, 3-phenylbenzyl and 4-methylbenzyl groups, and —OR is in β-orientation.

A lactose acceptor of formula 2B is also provided

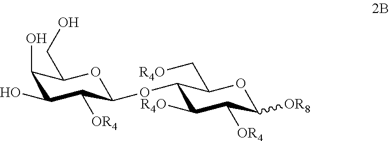

wherein R₄ is acyl and R₈ is a group removable by hydrogenolysis.

A compound of formula 2B above can be either an α- or β-anomer or an anomeric mixture of α- and β-anomers. The lactose acceptors of formula 2B can be crystalline solids, oils, syrups, precipitated amorphous material or spray dried products. If crystalline, a compound of formula 2B can exist either in an anhydrous or hydrated crystalline form by incorporating one or several molecules of water into its crystal structure. Likewise, a compound of formula 2B could exist as a crystalline substance, incorporating ligands such as organic molecules and/or ions into its crystal structure.

Preferably, R₄ in a compound of formula 2B is a low-migrating acyl group. More preferably, compounds of formula 2B wherein R₄ is a low-migrating acyl groups being a linear or branched chain alkanoyl group of 4 or more carbon atoms, especially 2-methyl-butyroyl or pivaloyl, or an unsubstituted or substituted benzoyl or naphthoyl group, especially benzoyl or 4-chlorobenzoyl, R₈ is benzyl optionally substituted with one or more of the following groups: phenyl, alkyl and halogen, particularly unsubstituted benzyl, 4-chlorobenzyl, 3-phenylbenzyl and 4-methylbenzyl groups, and —OR₈ is in β-orientation, are chosen.

Other features of the invention will become apparent in view of the following exemplary embodiments which are illustrative but not limiting of the invention.

### EXAMPLES

### Donors

Compounds of formula 1A (R₁═R₂═R₃=acetyl) and 1B (R₁═R₂═R₃═acetyl) were synthesized from N-acetyllactosamine peracetate and lacto-N-biose peracetate, respectively, according to the procedure described in Range et al. Tetrahedron 1997, 53, 1695.

### Example 1

### Benzyl 4-O-(6-O-tert-butyldimethylsilyl-β-D-galactopyranosyl)-β-D-glucopyranoside

A mixture of benzyl 2,3,6-tri-O-acetyl-4-O-(2,3-di-O-acetyl-β-D-galactopyranosyl)-β-D-glucopyranoside (103 g, Paulsen et al. Carbohydr. Res. 1985, 137, 39), tert-butyldimethylsilyl chloride (29 g) and imidazole (26.2 g) in DMF (473 ml) was stirred at room temperature for 24 hours. The reaction mixture was diluted with dichloromethane and the solvents were removed in vacuum. The residue was dissolved in dichloromethane (1 l) and extracted with 1 M sodium hydroxide solution (2×550 ml) and saturated sodium chloride solution (400 ml). The organic phase was dried over sodium sulphate and the solvents were evaporated. Hexane (300 ml) was then added to the syrupy residue and decanted. The remaining material was taken up in toluene (400 ml) and evaporated to dryness to give a thick syrup (138 g) which was used for the next step.

The above crude syrup (121.3 g) was dissolved in methanol (1.4 l) and the pH was adjusted to 9 by adding sodium methoxide (2.36 g). After stirring for 15 hours at room temperature the reaction mixture was diluted with dichloromethane and the solvents were evaporated. The residue was crystallized from diisopropyl ether-isopropanol mixture to give a white solid (70.16 g, 80% for two steps. A second crop (10.2 g, 12%) was obtained from the mother liquor.

### Example 2

### Benzyl 4-O-(3,4-O-isopropylidene-6-O-tert-butyldimethysilyl-β-D-galactopyranosyl)-β-D-glucopyranoside

Benzyl 4-O-(6-O-tert-butyldimethylsilyl-β-D-galactopyranosyl)-β-D-glucopyranoside (70 g) was dissolved in acetonitrile (245 ml) and 2,2-dimethoxypropane (88 ml). A 5% solution of tosic acid in water (80 ml) was added and the mixture was stirred at 45° C. for 2.5 hours. The acid was neutralized by adding Na₂CO₃ and the solvents were removed by evaporation. The residue was partitioned between chloroform (700 ml) and water (250 ml), and the organic phase was washed with water, 1M HCl-solution and brine. The residue obtained after drying and concentration was crystallized from tert-butyl methyl ether to give a white solid (47 g, 63%).

### Example 3

### Benzyl 2,3,6-tri-O-benzoyl-4-O-(2-O-benzoyl-3,4-O-isopropylidene-6-O-tert-butyldimethylsilyl-β-D-galactopyranosyl)-β-D-glucopyranoside

Benzyl 4-O-(3,4-O-isopropylidene-6-O-tert-butyldimethylsilyl-β-D-galactopyranosyl)-3-D-glucopyranoside (50.1 g) and 4-dimethylaminopyridine (0.21 g) were dissolved in pyridine (101 ml) and dichloromethane (80 ml). A solution of benzoyl chloride (48 ml) in dichloromethane (20 ml) was added slowly under cooling with cold water and the stirring was continued for 90 min. at room temperature. After quenching with methanol, the volatile solvents were removed by evaporation and the residue was taken up in dichloromethane (800 ml) which was washed with water, thoroughly with 10% citric acid solution and sat. Na₂CO₃-solution. The organic phase was dried and concentrated, and the residue was crystallized from tert-butyl methyl ether by adding hexane to give a white solid (77.8 g, 91%).

### Example 4

### Benzyl 2,3,6-tri-O-benzoyl-4-O-(2-O-benzoyl-β-D-galactopyranosyl)-β-D-glucopyranoside

A) Benzyl 2,3,6-tri-O-benzoyl-4-O-(2-O-benzoyl-3,4-O-isopropylidene-6-O-tert-butyldimethylsilyl-β-D-galactopyranosyl)-β-D-glucopyranoside (74.8 g) was suspended in a mixture of acetic acid (600 ml) and water (120 ml), and stirred at 65° C. for 2.5 hours. Appr. the half of the solvents was removed under vacuum when crystallization started. After cooling the crystals formed were collected and washed with ether. The mother liquor was concentrated to dryness and a second crop was crystallized from isopropanol. The two crops were combined to give a white solid (40 g, 63%).

M.p.: 213-214° C.

LC-MS (ES+): m/z 866.4 [M+NH₄]⁺, 871.4 [M+Na]⁺, 887.5 [M+K]⁺

¹³C-NMR (400 MHz, CDCl₃) δ: Glc C-1 98.8 C-2 71.6 C-3 73.4 C-4 76.5 C-5 73.0 C-6 62.8, Gal C-1 101.1 C-2 73.6 C-3 72.8 C-4 69.7 C-5 74.3 C-6 62.1, CH₂Ph 70.3, CH₂Ph 136.5 128.5 128.1 128.0, COPh 166.4, 166.0 165.6, 165.2, COPh 136.4, 133.3 133.2, 129.8 129.7 129.6 129.2, 129.1 128.6.

B) Benzyl 4-O-(3-O-allyl-β-D-galactopyranosyl)-β-D-glucopyranoside (Jung et al. Liebigs Ann. Chem., 1989, 1099; 33.07 g), benzaldehyde dimethylacetal (12.6 ml) and p-toluenesulfonic acid (1.33 g) were dissolved in DMF (420 ml). The solution was kept at 70° C. for 45 min. A second part of benzaldehyde dimethylacetal (12.6 ml) was then added and the heating was continued for additional 30 min. After cooling solid NaHCO₃ (600 mg) was added for neutralizing the acid. The solvents were evaporated under vacuum giving a white solid crude which was used in the next step without further purification.

To a cold solution of the above crude material in pyridine (280 ml) benzoyl chloride (42 ml) and 4-dimethylaminopyridine (0.20 g) were added. The reaction mixture was stirred for 24 hours at room temperature then evaporated to dryness. The residue was dissolved in chloroform (1000 ml) and extracted with saturated NaHCO₃-solution. The organic phase was dried over Na₂SO₄ and evaporated. After adding ether a white powder was precipitated (47 g, 69% for two steps).

The above white powder was suspended in 80% acetic acid solution (2 l) and the solution was refluxed for 1 h giving a clear solution. The solvents were completely evaporated and the residue was treated with ether to precipitate a white powder, which, after filtration and drying, was suspended in methanol (1800 ml). A suspension of Pd on charcoal (10%, 5 g) in water (100 ml) was carefully added followed by the addition of p-toluenesulfonic acid (5 g) in water (100 ml). The reaction mixture was refluxed for 14 hours. The catalyst was filtered off and washed with CHCl₃. The solvents were evaporated and the residue was flash-chromatographed on silica. The clear fractions were collected and evaporated giving the product which was precipitated by addition of ether (20 g, 49% in two steps).

### Example 5

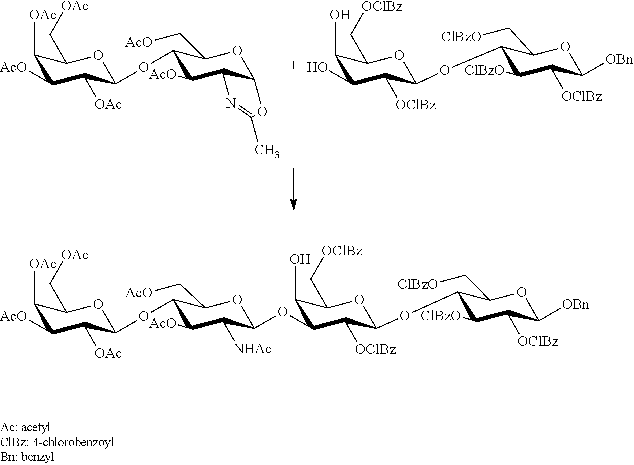

Acceptor was made in accordance with Example 2 of WO 2011/100980.

To a mixture of donor (1.823 g, 2.95 mmol) and acceptor (2.076 g, 1.85 mmol) in dry toluene (15 mL), BF₃.OEt₂ (105 μL, 0.83 mmol)) was added. The reaction flask was heated to 60° C. for 5 hours when Et₃N (100 μL) was added whereupon the mixture was allowed to reach RT and then evaporated to get a foam. The obtained crude product was purified by flash chromatography and re-crystallized to get the product as a white solid (1.65 g, 0.95 mmol, 51%). M.p. 254.5-255.5° C.

¹H-NMR (CDCl₃, 600 MHz) δ=1.42 (s, 3H), 1.93 (s, 3H), 1.96 (s, 3H), 1.98 (s, 3H), 1.99 (s, 3H), 2.00 (s, 3H), 2.01 (s, 3H), 2.80 (s, 1H), 3.47 (dd, 1H, J=5.1 5.1 Hz), 3.49 (ddd, 1H, J=2.6 4.9, 9.3 Hz), 3.58 (ddd, 1H, J=8.0, 8.7, 9.8 Hz), 3.62 (dd, 1H, J=9.1 9.3 Hz), 3.66 (dd, 1H, J=3.1 9.7 Hz), 3.68 (ddd, 1H, J=1.8 4.4 9.8 Hz), 3.72 (dd, 1H, J=5.1 11.6 Hz), 3.82 (dd, 1H, J=6.3 7.1 Hz), 3.90 (d, 1H, J=3.1 Hz), 3.97 (dd, 1H, J=4.9 12.2 Hz), 4.02 (dd, 1H, J=7.1 11.2 Hz), 4.03 (dd, 1H, J=9.0 9.8 Hz), 4.06 (dd, 1H, J=6.3 11.2 Hz), 4.20 (dd, 1H, J=5.1 11.6 Hz), 4.38 (dd, 1H, J=1.8 12.1 Hz), 4.43 (dd, 1H, J=2.6 12.2 Hz), 4.44 (d, 1H, J=8.0 Hz), 4.48 (dd, 1H, J=4.4, 12.1 Hz), 4.50 (d, 1H, J=8.2 Hz), 4.52 (d, 1H, J=12.6 Hz), 4.63 (d, 1H, J=8.0 Hz), 4.65 (d, 1H, J=8.0 Hz), 4.77 (d, 1H, J=12.6 Hz), 4.92 (dd, 1H, J=3.2 9.9 Hz), 5.03 (dd, 1H, J=9.1 9.8 Hz), 5.04 (dd, 1H, J=8.0 9.9 Hz), 5.14 (d, 1H, J=8.7 Hz), 5.30 (d, 1H, J=3.2 Hz), 5.31 (dd, 1H, J=8.2 9.7 Hz), 5.36 (dd, 1H, J=8.0 9.5 Hz), 5.54 (dd, 1H, J=9.0 9.5 Hz), 7.08-8.0 (m, 25H).

¹³C-NMR (CDCl₃, 150.9 MHz) δ=20.5, 20.6 (3C), 20.7 (2C), 22.6, 54.7, 60.7, 61.5, 62.8, 62.9, 66.5, 67.6, 69.1, 70.6, 70.7, 70.8, 71.2, 71.6, 71.9, 72.3, 72.8, 72.9, 73.0, 75.4, 80.3, 98.8, 100.5, 100.7, 101.0, 127.3, 127.6, 127.7, 128.0, 128.2, 128.4, 128.6, 128.7, 128.9, 129.1, 129.2, 130.8, 130.9, 131.0, 131.1, 131.2, 136.3, 136.3, 163.6, 164.4, 164.6, 165.1, 165.2, 169.1, 170.0, 170.1, 170.2, 170.3 (2C), 170.4.

### Example 6

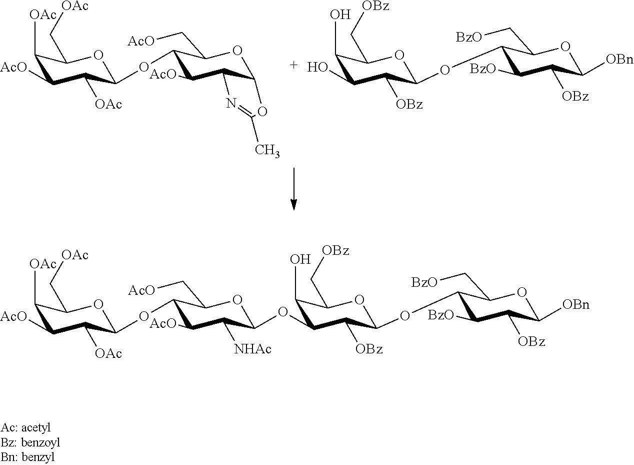

Acceptor was made in accordance with: Example 4 of WO 2011/100980.

A) To a mixture of donor (0.488 g, 0.789 mmol) and acceptor (0.470 g, 0.493 mmol) in a dry toluene/DCM mixture (9:1, 5 mL), BF₃.OEt₂ (30 μL, 0.24 mmol)) was added. The reaction flask was heated to 55° C. over night and quenched with Et₃N. The mixture was allowed to reach RT and then evaporated to get the crude product. Purification by flash chromatography and re-crystallization gave the product as a white solid (0.387 g, 0.2465 mmol, 50%). M.p. 255-256° C.

¹H-NMR (CDCl₃, 300 MHz) δ=1.40 (s, 3H), 1.94 (s, 3H), 1.96 (s, 3H), 1.98 (s, 3H), 2.00 (s, 3H), 2.02 (s, 3H), 2.11 (s, 3H), 2.65 (s, 1H), 3.45-3.51 (m, 2H), 3.58-4.21 (m, 11H), 4.39-4.44 (m, 3H), 4.50-4.55 (m, 3H), 4.64 (d, 1H, J=8.1 Hz), 4.64 (d, 1H, J=8.1 Hz), 4.78 (d, 1H, J=12.6 Hz), 4.91 (dd, 1H, J=3.3 10.2 Hz), 4.99-5.07 (m, 3H), 5.30 (d, 1H, J=3.3 Hz), 5.36 (dd, 1H, J=8.1 9.6 Hz), 5.45 (dd, 1H, J=7.8 9.9 Hz), 5.63 (dd, 1H, J=9.6 9.6 Hz), 7.09-8.09 (m, 30H).

¹³C-NMR (CDCl₃, 75.4 MHz) δ 20.4, 20.5, 20.6 (2C), 20.6, 20.7, 22.4, 54.8, 60.6, 61.7, 62.5, 62.7, 66.5, 67.8, 69.0, 70.4, 70.5, 70.8, 71.0, 71.7, 71.8, 72.4, 72.70 (2C), 72.9, 75.7, 76.1, 80.4, 99.0, 100.6, 100.7, 100.9, 127.6-129.9, 132.9, 133.1, 133.2, 133.3, 133.5, 136.4, 164.5, 165.2, 165.4, 165.9, 166.0, 169.1, 170.0, 170.0, 170.2, 170.3, 170.6.

B) To a solution of the acceptor (135.2 g) in toluene (400 ml) and dichloromethane (120 ml) BF₃-etherate (16.5 ml) was added at 75° C. followed by the addition of a solution of the donor (132 g dissolved in 300 ml of toluene and 100 ml of dichloromethane). After the addition the reaction mixture was stirred for 2 hours at 75° C., cooled to room temperature, diluted with dichloromethane (540 ml) and extracted with sat. sodium bicarbonate solution (140 ml). The aqueous phase was extracted with dichloromethane (150 ml) and the combined organic phases were washed with brine (150 ml). The organic phase was subjected to distillation whereupon approx. 650 ml of solvents were distilled off. Toluene (450 ml) was then added and the distillation was continued until a crystalline slurry was obtained. After cooling, filtration and drying the title compound was obtained as a white crystalline material (156.7 g, 75%).

### Example 7

### 1-O-benzyl-β-LNnT

The compound of example 6 (13.6 g) was suspended in methanol (200 ml) and a solution of NaOMe (25% in methanol, 2.7 ml) was added. The suspension is stirred at 55-56° C. for 7 hours. Approx. 45-50 ml of methanol was distilled off and the resulting slurry was stirred at room temperature for 7 hours, then filtered, and washed with methanol. The white solid obtained was then dried in vacuo to yield 5.75 g (83%) of the title compound. Characteristics were in accordance with those disclosed in WO 2011/100980.

### Example 8

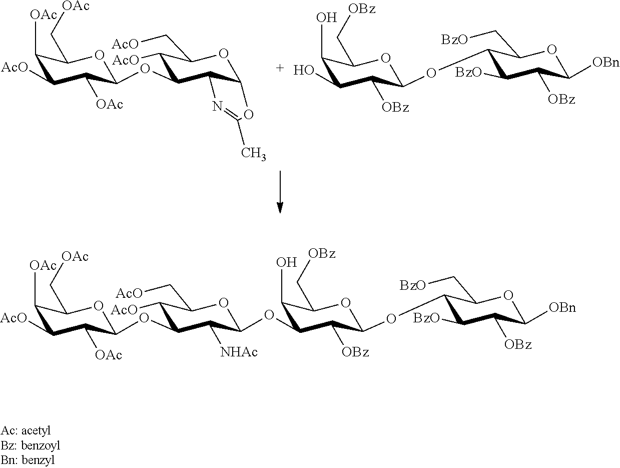

Acceptor was made in accordance with: Example 4 of WO 2011/100980.

The donor (100 mg) and the acceptor (200 mg) were suspended in toluene (1 ml), powdered molecular sieves 4A (100 mg) were added followed by BF₃.OEt₂ (10 μl). The mixture was shaked in a closed vial at 73-75° C. for 12 hours and, after cooling was brought directly to a column of silica gel. Chromatography gave 108 mg of white solid (42%) yield.

MS: m/z calculated for Co₈₀H₈₃NO₃₂ 1569.49. found 1570.58 [M+H]⁺ and 1592.7 [M+Na]⁺.

¹H NMR (300 MHz, CDCl₃): δ 7.85-8.1 (m, 10H), 7.05-7.65 (m, 20H), 5.63 (overlapping t, J=9.3 Hz, 1H and br. s, 1H, NH), 5.47 (dd, J=9.7, 7.8 Hz, 1H), 5.35 (dd, J=9.7, 7.9 Hz, 1H), 5.26 (d, J=3.5 Hz, 1H), 5.05 (d, J=8.1 Hz, 1H), 4.96 (dd, J=10.4, 7.8 Hz, 1H), 4.75-4.85 (m, 3H), 4.65 (d, J=7.8 Hz, 1H), 4.4-4.6 (m, 5H), 4.30 (d, J=7.8 Hz, 1H), 4.1-4.2 (m, 2H), 3.95-4.05 (m, 4H), 3.94 (d, J=3.5 Hz, 1H), 3.45-3.8 (m, 7H), 2.95 (dt, J=10.1, 7.4 Hz, 1H, CH—NHAc), 2.09, 2.01, 1.97, 1.95, 1.93, 1.92 (6 s, 18H, 6 Me of OAc), 1.25 (s, 3H, Me of NHAc).

¹³C NMR (75 MHz, CDCl₃=77.16 ppm) δ 171.18, 170.67, 170.44, 170.27, 170.20, 169.41, 168.96 (7 C═O of Ac), 166.12, 166.04, 165.77, 165.30, 165.03 (5 C═O of Bz), 136.59 (Bn), 133.73, 133.54, 133.42, 133.28, 133.14 (5 Bz), 129.95, 129.88, 129.62, 129.41, 129.12, 128.84, 128.78, 128.60, 128.40, 128.30, 127.95, 127.80 (aromatic CH), 125.39, 100.83, 100.65, 99.39, 99.22 (4 anomeric CH), 80.89, 76.04, 75.76, 73.06, 72.91, 72.45, 72.00, 71.85, 71.12, 71.05, 70.60 (CH₂OBn), 69.39, 68.89, 68.02, 66.90, 62.72, 62.64, 62.31, 61.06, 58.47, 22.68 (Me of NHAc), 20.84, 20.75, 20.72, 20.63 (Me of OAc, overlapping).

### Example 9

### 1-O-benzyl-β-LNT

The compound according to example 8 was deacylated under the condition disclosed in example 7 providing the title compound as a white solid.

¹H-NMR (D₂O, 400 MHz) δ 2.03 (s, 3H, CH₃CONH), 3.35 (dd, 1H, J=8.1 8.5 Hz, H-2), 3.49 (m, 1H, H-5″), 3.53 (m, H-2′″), 3.65 (m, 1H, H-3′″), 3.57 (dd, 1H, J=8.1 9.0 Hz, H-4″), 3.58 (m, 1H, H-5), 3.59 (dd, 1H, J=7.7 10.0 Hz, H-2′), 3.62 (m, 1H, H-3), 3.63 (m, 1H, H-4), 3.71 (m, 1H, H-5′), 3.71 (m, 1H, H-5′″), 3.73 (dd, 1H, J=3.3 10.0 Hz, H-3′), 3.76 (m, 2H, H-6ab′″), 3.76 (m, 2H, H-6ab′), 3.80 (m, 1H, H-6a″), 3.80 (dd, 1H, J=5.0 12.2 Hz, H-6a), 3.82 (dd, 1H, J=8.1 10.5 Hz, H-3″), 3.90 (m, 1H, H-6b″), 3.90 (dd, 1H, J=8.4 10.5 Hz, H-2″), 3.92 (d, 1H, J=3.3 Hz, H-4′″), 3.98 (dd, 1H, J=1.6 12.2 Hz, H-6b), 4.15 (d, 1H, J=3.3 Hz, H-4′), 4.44 (d, 1H, J=7.7 Hz, H-1′), 4.45 (d, 1H, J=7.7 Hz, H-1′″), 4.56 (d, 1H, J=8.1 Hz, H-1), 4.73 (d, 1H, J=8.4 Hz, H-1″), 4.76 (d, 1H, J=11.7 Hz, CH₂Ph), 4.94 (d, 1H, J=11.7 Hz, CH₂Ph), 7.40-7.50 (m, 5H, Ph).

¹³C-NMR (D₂O, 100 MHz) δ 24.9 (CH₃CONH), 57.4 (C-2″), 62.8 (C-6), 63.2 (C-6″), 63.7 (C-6′″), 63.7 (C-6′), 71.0 (C-4′), 71.2 (C-4′″), 71.3 (C-4″), 72.7 (C-2′), 73.4 (C-2′″), 74.2 (CH₂Ph), 75.2 (C-3′″), 75.5 (C-2), 77.1 (C-3), 77.5 (C-5′), 77.6 (C-5′″), 77.9 (C-5), 78.0 (C-5″), 81.1 (C-4), 84.7 (C-3′), 84.8 (C-3″), 103.7 (C-1), 105.3 (C-1″), 105.6 (C-1′), 106.2 (C-1′″), 131.1 (Ph), 131.4 (2C, Ph), 131.5 (2C, Ph), 139.2 (Ph), 177.7 (CH₃CONH).

M.p. 245° C. (dec.). [α]_{D}22=−10.3 (c=1, H₂O).

### Example 10

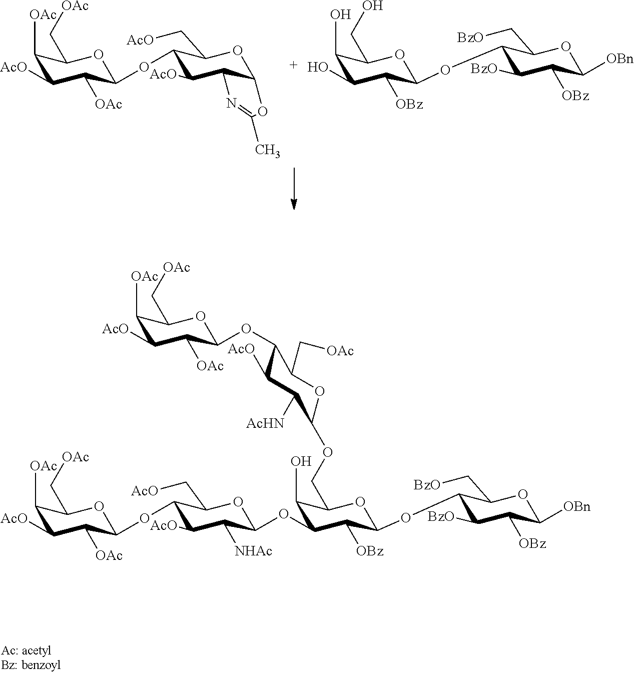

The compound according to example 4 (47.8 g) was dissolved in THF (100 ml) and BF₃-etherate was added (0.5 ml). To this solution the donor (98.8 g) dissolved in toluene (260 ml) was added very slowly (4 days) at 68-75° C. After cooling to room temperature sat. sodium bicarbonate solution (60 ml) and water (30 ml) were added and the resulting biphasic system was extracted with ethyl acetate (200 ml). The organic phase was then washed with brine and water, dried over sodium sulphate and evaporated. The residue was silylated with TBDMS chloride to derivatize the polar by-product(s). After column chromatography the title hexasaccharide was isolated (105.9 g).

¹H and ¹³C resonance assignments in CDCl₃, 25° C., 600 MHz:

Carbonyls of the O-acyl protective groups: 170.6 170.6 170.5 170.5 170.3 170.3 170.1 170.0 170.0 170.0 169.3 169.1 (OAc) 165.9 165.5 165.3 164.6 (OBz)

| Ring | Proton | Shift (ppm) | Mult. | J (Hz) | Carbon | Shift (ppm) |
|---|---|---|---|---|---|---|
| Galβ1-4GlcNAcβ1- | H-1 | 4.67 | d | 7.6 | C-1 | 98.8 |
| 3(Galβ1-4GlcNAcβ1- | CH_{2X}-Ph | 4.76 | d | 12.4 | CH₂-Ph | 70.4 |
| 6)Galβ1-4Glc | CH_{2Y}-Ph | 4.53 | d | 12.4 | | |
| | CH₂-Ph | 7.02-7.18 | m | | CH₂-Ph | 127.7, 127.8, 128.3 |
| | H-2 | 5.40 | dd | 9.2, 7.6 | C-2 | 71.8 |
| | H-3 | 5.49 | dd | 9.2, 9.0 | C-3 | 74.1 |
| | H-4 | 4.14 | dd | 9.0, 8.4 | C-4 | 75.6 |
| | H-5 | 3.71 | m | | C-5 | 72.9 |
| | H-6x | 4.54 | dd | 12.5, 2.2 | C-6 | 62.6 |
| | H-6y | 4.33 | dd | 12.5, 5.2 | | |
| Galβ1-4GlcNAcβ1- | H-1 | 4.53 | d | 8.1 | C-1 | 100.7 |
| 3(Galβ1-4GlcNAcβ1- | H-2 | 5.34 | dd | 8.3, 8.1 | C-2 | 71.2 |
| 6)Galβ1-4Glc | H-3 | 3.59 | dd | 8.3, 3.0 | C-3 | 80.3 |
| | H-4 | 3.85 | d | 3.0 | C-4 | 67.6 |
| | H-5 | 3.19 | m | | C-5 | 73.0 |
| | H-6x | 3.53 | m | | C-6 | 67.9 |
| | H-6y | 3.07 | m | | | |
| Galβ1-4GlcNAcβ1- | H-1 | 4.54 | d | 8.8 | C-1 | 101.2 |
| 3(Galβ1-4GlcNAcβ1- | H-2 | 3.66 | dd | 9.0, 8.8 | C-2 | 54.1 |
| 6)Galβ1-4Glc | O = CNH | 5.04 | s | | | |
| | O = CCH₃ | 1.37 | s | | O = CCH₃ | 22.4 |
| | | | | | O = CCH₃ | 170.4 |
| | H-3 | 4.94 | dd | 10.4, 9.0 | C-3 | 72.1 |
| | H-4 | 3.63 | dd | 10.4, 8.7 | C-4 | 76.1 |
| | H-5 | 3.51 | m | | C-5 | 72.7 |
| | H-6x | 4.35 | dd | 11.8, 2.0 | C-6 | 62.0 |
| | H-6y | 4.04 | dd | 11.8, 6.0 | | |
| Galβ1-4GlcNAcβ1- | H-1 | 4.42 | d | 8.7 | C-1 | 100.9 |
| 3(Galβ1-4GlcNAcβ1- | H-2 | 5.03 | dd | 10.2 | C-2 | 69.1 |
| 6)Galβ1-4Glc | H-3 | 4.91 | dd | 10.2, 3.0 | C-3 | 70.8 |
| | H-4 | 5.29 | d | 3.0 | C-4 | 66.5 |
| | H-5 | 3.81 | m | | C-5 | 70.6 |
| | H-6x | 4.03 | m | | C-6 | 60.7 |
| | H-6y | | | | | |
| Galβ1-4GlcNAcβ1- | H-1 | 3.88 | m | | C-1 | 101.3 |
| 3(Galβ1-4GlcNAcβ1- | H-2 | 3.87 | m | | C-2 | 53.3 |
| 6)Galβ1-4Glc | O = CNH | 5.86 | d | 8.1 | | |
| | O = CCH₃ | 1.96 | s | | O = CCH₃ | 23.3 |
| | | | | | O = CCH₃ | 170.2 |
| | H-3 | 5.00 | dd | 10.0, 8.9 | C-3 | 72.6 |
| | H-4 | 3.71 | dd | 8.9, 8.7 | C-4 | 76.1 |
| | H-5 | 3.44 | m | | C-5 | 72.6 |
| | H-6x | 4.37 | dd | 11.4, 2.0 | C-6 | 62.5 |
| | H-6y | 4.03 | dd | 11.4, 5.4 | | |
| Galβ1-4GlcNAcβ1- | H-1 | 4.48 | d | 7.9 | C-1 | 101.1 |
| 3(Galβ1-4GlcNAcβ1- | H-2 | 5.11 | dd | 9.7, 7.9 | C-2 | 69.1 |
| 6)Galβ1-4Glc | H-3 | 4.96 | dd | 9.7, 3.0 | C-3 | 70.9 |
| | H-4 | 5.34 | d | 3.0 | C-4 | 66.6 |
| | H-5 | 3.87 | m | | C-5 | 70.7 |
| | H-6x | 4.11 | m | | C-6 | 60.8 |
| | H-6y | | | | | |

### Example 11

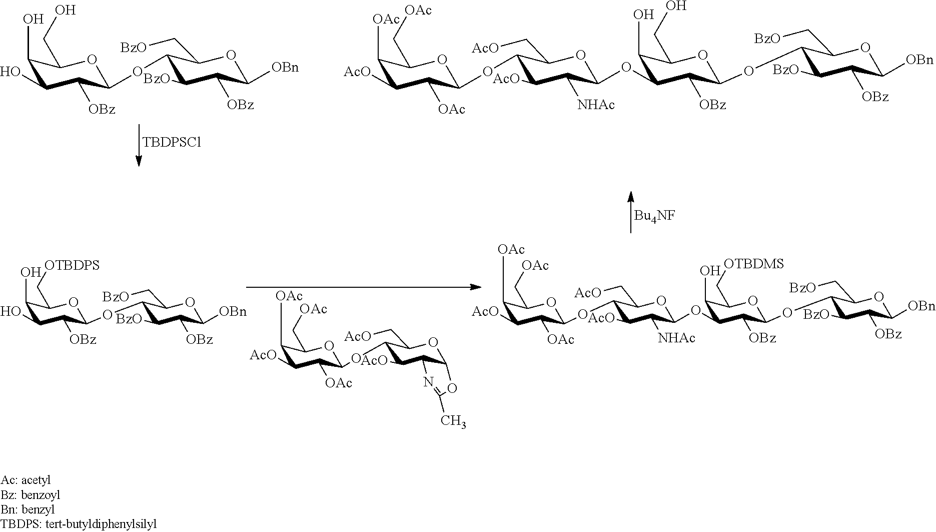

Benzyl 2,3,6-tri-O-benzoyl-4-O-(2-O-benzoyl-β-D-galactopyranosyl)-1-D-glucopyranoside (10 g), 4-dimethylamino-pyridine (30 mg) and tert-butyldiphenylsilyl chloride (3.1 ml) were dissolved in pyridine (50 ml), and the mixture was stirred for 20 hours at 45° C. After evaporating the solvent, the residue was taken up in dichloromethane (120 ml), extracted with water, 10% citric acid solution and sat. sodium bicarbonate solution, followed by drying and removal of the solvent. Hexane was added to the residue obtained and the precipitation was filtered, washed with isopropyl ether and dried to give benzyl 2,3,6-tri-O-benzoyl-4-O-(2-O-benzoyl-6-O-tert-butyldiphenylsilyl-β-D-galactopyranosyl)-β-D-glucopyranoside as a solid (11.42 g).

10 g of the above solid was dissolved in a mixture of toluene (30 ml) and dichloromethane (10 ml). To this solution BF₃-etherate (180 μl) was added followed by the addition of a solution of the donor (8.5 g) in toluene (26 ml) at 82° C. during 6 hours. The stirring was continued at the same temperature for additional 2 hours. After cooling to room temperature the reaction mixture was diluted with dichloromethane (60 ml), extracted with sat. sodium bicarbonate solution and brine, and dried. After evaporating the solvents the crude sialylated tetrasaccharide was obtained as a yellow foam (23.5 g), from which 19 g was purified by column chromatography to give the pure sialylated tetrasaccharide (6.8 g).

6.25 g of the above silylated tetrasaccharide was dissolved in THF (28 ml) and Bu₄NF (1.15 g) was added. The mixture was stirred at room temperature for 20 min., diluted with dichloromethane (130 ml) and washed with sat. sodium bicarbonate solution. The organic phase was dried and evaporated, the obtained foam was solidified by adding hexane to give the title tetrasaccharide diol as powder (5.55 g).

### Example 12

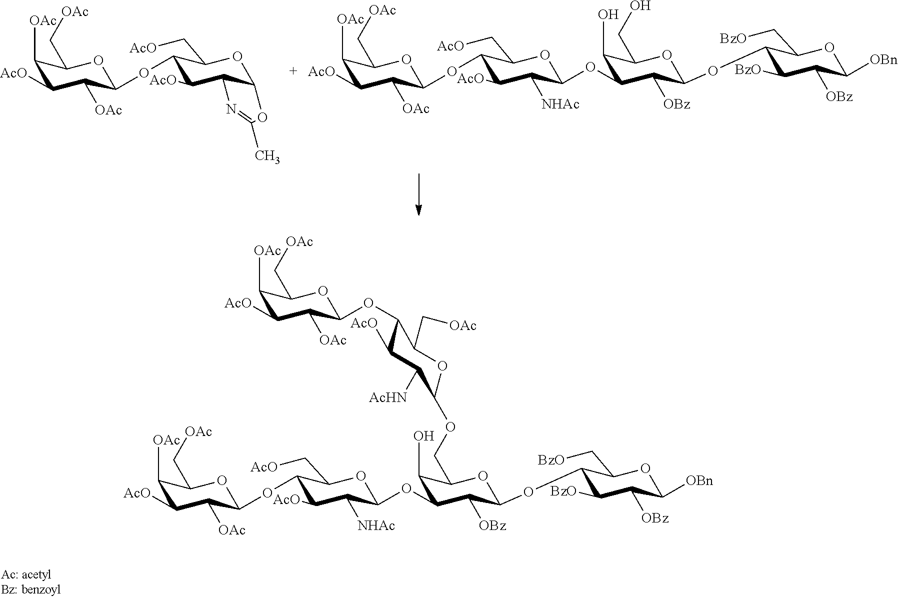

The compound according to example 11 (8.0 g) was dissolved in a mixture of toluene (24 ml) and dichloromethane (8 ml), and BF₃-etherate was added (67 μl). To this solution the donor (5.05 g) dissolved in toluene (15 ml) was added over 6 hours at 75° C. After cooling to room temperature the mixture was diluted with dichloromethane (100 ml), washed with sat. sodium bicarbonate solution and brine (30 ml), dried and evaporated to give a foamy residue (13.4 g). A small sample was chromatograhed to obtain the pure title tetrasaccharide having identical analytical data with those of compound of example 10.

### Example 13

### 1-O-benzyl-3-LNnH

The compound according example 10 (5.71 g) was deacylated with NaOMe/MeOH as described in example 19 giving the title compound (1.99 g).

LC-MS (ES+): m/z 1185.5 [M+Na]⁺, 604.3 [M+2Na]²⁺

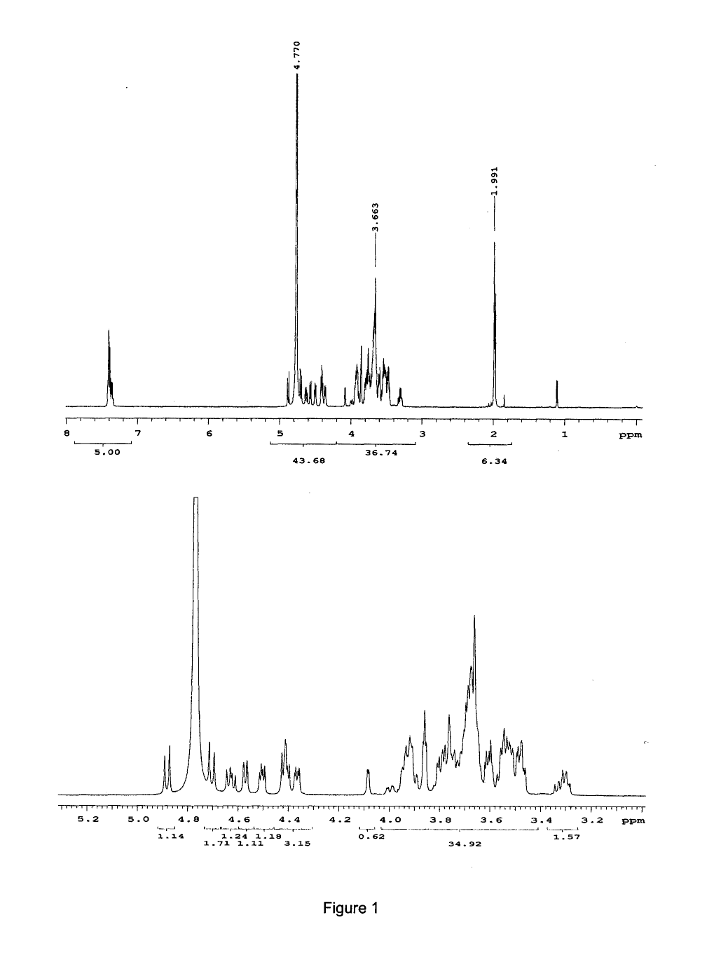

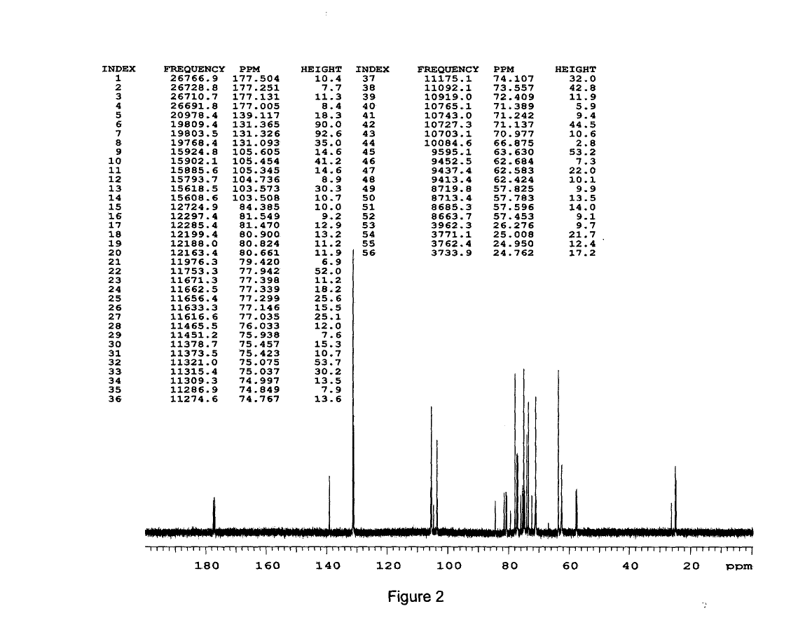

NMR: see FIGS. 1 and 2.

### Example 14

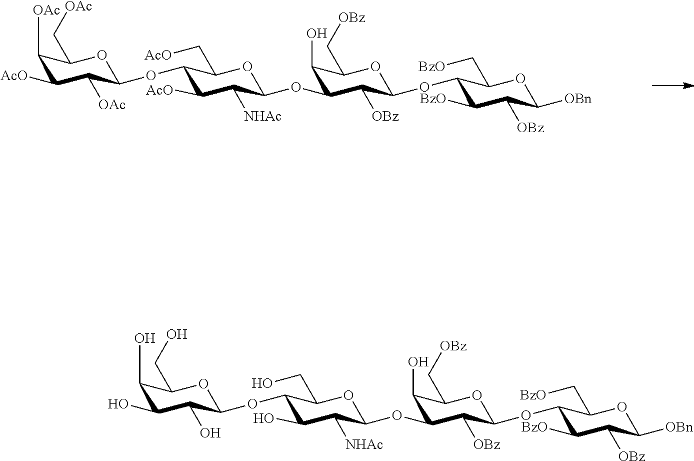

The compound according to example 6 (58.5 g) was dissolved in dichloromethane (300 ml) and methanol (300 ml). To this solution acetyl chloride (19 ml) was added dropwise in 1 hour while keeping the temperature at −5° C. After 30 min. additional stirring the cooling bath was removed and the stirring was continued overnight at room temperature. Sodium bicarbonate was added to adjust the pH to 7, then the mixture was diluted with methanol (50 ml) and dichloromethane (50 ml) followed by the addition of charcoal. After filtration the filtrate was evaporated to half of its original volume and the solution turned into a suspension. After dilution with methanol the solid was filtered off and dried to give a white powder (39.2 g, 80%).

LC-MS (ES+): m/z 1318 [M+H]³⁰, 1340.6 [M+Na]⁺

### Example 15

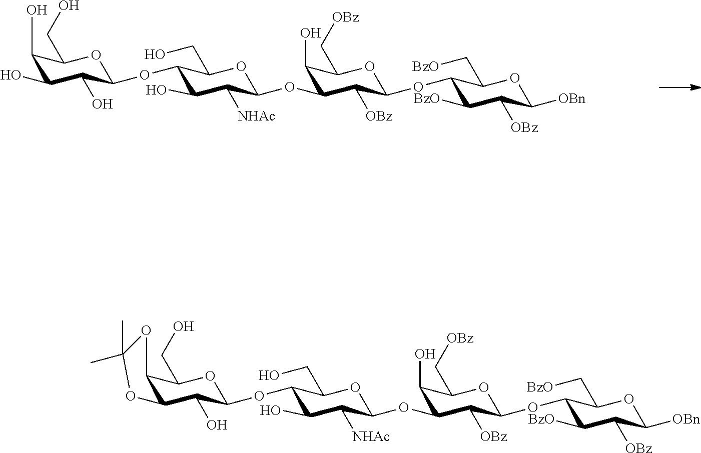

A) To a solution of the compound according to example 14 (9.8 g) in acetonitrile (49 ml) 2,2-dimethoxy-propane (5.0 ml) and a 10% solution of p-toluenesulfonic acid monohydrate in acetonitrile (395 μl) were added. The mixture stirred at 45° C. for 1 hour. After cooling to room temperature the acid was neutralized by adding solid sodium bicarbonate. After removing the solvent, the residue was taken up in dichloromethane (140 ml) and water (20 ml), the phases were separated, the organic phase was washed with water and dried. After removal the solvent the title compound was obtained as a beige solid material (9.9 g).

¹H and ¹³C resonance assignments in DMSO, 25° C., 400 MHz (acyl protecting groups are not detailed in the table):

| Ring | Proton | Shift (ppm) | Mult. | J (Hz) | Carbon | Shift (ppm) |
|---|---|---|---|---|---|---|
| Galβ1-4GlcNAcβ1-3Galβ1-4Glc | H-1 | 5.06 | d | 8.0 | C-1 | 98.8 |
| | CH_{2X}-Ph | 4.66 | d | 12.9 | CH₂-Ph | 70.2 |
| | CH_{2Y}-Ph | 4.50 | d | 12.9 | | |
| | CH₂-Ph | 7.03-7.22 | m | | CH₂-Ph | 128.0, 127.5, 127.2 |
| | H-2 | 5.18 | dd | 9.5, 8.0 | C-2 | 72.2 |
| | H-3 | 5.66 | dd | 9.5, 9.3 | C-3 | 72.2 |
| | H-4 | 4.24 | dd | 9.5, 9.3 | C-4 | 75.3 |
| | H-5 | 3.98 | m | | C-5 | 72.1 |
| | H-6x | 4.44 | m | | C-6 | 62.6 |
| | H-6y | | | | | |
| Galβ1-4GlcNAcβ1-3Galβ1-4Glc | H-1 | 4.71 | d | 8.1 | C-1 | 100.5 |
| | H-2 | 5.15 | dd | 10.0, 8.1 | C-2 | 70.3 |
| | H-3 | 3.79 | m | | C-3 | 80.7 |
| | H-4 | 4.14 | m | | C-4 | 67.1 |
| | H-5 | 3.70 | m | | C-5 | 71.8 |
| | H-6x | 4.09 | dd | 10.7, 5.5 | C-6 | 62.8 |
| | H-6y | 3.72 | dd | 10.7, 4.0 | | |
| Galβ1-4GlcNAcβ1-3Galβ1-4Glc | H-1 | 4.54 | d | 8.5 | C-1 | 101.9 |
| | H-2 | 3.29 | m | | C-2 | 55.0 |
| | O = CNH | 4.38 | d | 2.6 | | |
| | O = CCH₃ | 0.89 | s | | O = CCH₃ | 13.0 |
| | | | | | O = CCH₃ | 168.2 |
| | H-3 | 3.24 | m | | C-3 | 74.4 |
| | H-4 | 3.29 | m | | C-4 | 80.6 |
| | H-5 | 3.45 | m | | C-5 | 71.7 |
| | H-6x | 3.60 | m | | C-6 | 60.0 |
| | H-6y | | | | | |
| Galβ1-4GlcNAcβ1-3Galβ1-4Glc | H-1 | 4.19 | d | 8.1 | C-1 | 102.9 |
| | H-2 | 3.18 | ddd | 8.1, 7.7, 5.3 | C-2 | 72.4 |
| | OH | 5.41 | d | 5.3 | | |
| | H-3 | 3.95 | dd | 7.0, 5.7 | C-3 | 79.2 |
| | H-4 | 4.07 | dd | 5.7, 2.2 | C-4 | 73.1 |
| | >C(CH₃)₂ | 1.19 | s | | >C(CH₃)₂ | 26.2 |
| | >C(CH₃)₂ | 1.41 | s | | >C(CH₃)₂ | 28.0 |
| | | | | | >C(CH₃)₂ | 108.6 |
| | H-5 | 3.80 | m | | C-5 | 73.3 |
| | H-6x | 3.54 | m | | C-6 | 60.3 |
| | H-6y | 3.48 | m | | | |
| | OH | 4.81 | t | 5.2 | | |

B) A mixture of compound according to example 14 (33.3 g), 4-dimethylaminopyridine (120 mg) and tert-butyldiphenylsilyl chloride (14 ml) in pyridine (130 ml) was stirred for 5 hours at room temperature. After addition of extra tert-butyldiphenylsilyl chloride (3.5 ml) the stirring was continued for 16 hours. Most of the pyridine was evaporated under vacuum, the residue was dissolved in dichloromethane (500 ml) and washed with water, 10% citric acid solution, sat. sodium bicarbonate solution and brine. After removal of the solvent the resulting thick syrup was solidified by adding hexane giving the disilylated product as a beige solid (50.4 g).

The above beige material was dissolved in acetonitrile (250 ml) and isopropylidenated as described in method A (using 30 ml of 2,2-dimethoxypropane and 0.24 g of p-toluenesulfonic acid monohydrate). The product was purified by column chromatography to give 23.2 g of isopropylidenated product.

The above material in tetrahydrofuran (200 ml) was treated with tetrabutylammonium fluoride (7.45 g) at room temperature. After completion of the reaction (1 hour) the solvent was removed in vacuo, the residue was dissolved in chloroform (300 ml), washed with sat. sodium bicarbonate solution and brine, and dried. After removal of the solvent the residue was precipitated in hexane obtaining the title material as a beige solid (18.8 g).

### Example 16

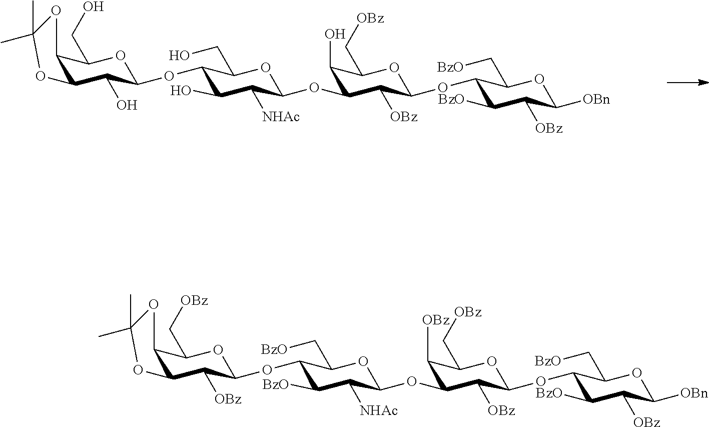

To a mixture of compound according to example 15 (0.62 g), 4-dimethylamino-pyridine (1 mg) in pyridine (4.5 ml) benzoyl chloride (355 μl) was added at 0° C. The mixture was stirred at room temperature for 21 hours, then additional portion of benzyl chloride (100 μl) was dropped in. After 18 hours methanol (1 ml) was slowly added followed by addition of toluene (401 ml), and the mixture was evaporated to dryness. The residue was dissolved in dichloromethane (25 ml), extracted with water and sat. sodium bicarbonate solution and dried. After removing the solvent the resulting material was solidified in hexane to give the title compound (0.66 g).

LC-MS (ES+): m/z 946.2 [M+2Li]²⁺

¹H and ¹³C resonance assignments in CDCl₃, 25° C., 400 MHz (acyl protecting groups are not detailed in the table):

| Ring | Proton | Shift (ppm) | Mult. | J (Hz) | Carbon | Shift (ppm) |
|---|---|---|---|---|---|---|
| Galβ1-4GlcNAcβ1-3Galβ1-4Glc | H-1 | 4.66 | d | 7.9 | C-1 | 99.1 |
| | CH_{2X}-Ph | 4.80 | d | 12.5 | CH₂-Ph | 70.5 |
| | CH_{2Y}-Ph | 4.56 | d | 12.5 | | |
| | CH₂-Ph | 7.07-7.22 | m | | CH₂-Ph | 128.4, 128.0, 127.8 |
| | H-2 | 5.49 | dd | 9.6, 7.9 | C-2 | 71.5 |
| | H-3 | 5.63 | dd | 9.6, 9.5 | C-3 | 72.6 |
| | H-4 | 4.15 | m | | C-4 | 77.1 |
| | H-5 | 3.74 | m | | C-5 | 72.9 |
| | H-6x | 4.53 | dd | 12.1, 1.7 | C-6 | 62.5 |
| | H-6y | 4.41 | dd | 12.1, 4.4 | | |
| Galβ1-4GlcNAcβ1-3Galβ1-4Glc | H-1 | 4.60 | d | 7.9 | C-1 | 100.5 |
| | H-2 | 5.48 | dd | 9.6, 7.9 | C-2 | 71.3 |
| | H-3 | 3.78 | m | | C-3 | 78.5 |
| | H-4 | 5.46 | m | | C-4 | 69.1 |
| | H-5 | 3.61 | m | | C-5 | 71.2 |
| | H-6x | 4.19 | dd | 11.7, 5.2 | C-6 | 62.8 |
| | H-6y | 3.78 | m | | | |
| Galβ1-4GlcNAcβ1-3Galβ1-4Glc | H-1 | 4.69 | d | 8.2 | C-1 | 101.2 |
| | H-2 | 3.69 | ddd | 9.5, 8.7, 8.2 | C-2 | 55.0 |
| | O = CNH | 5.08 | d | 8.7 | | |
| | O = CCH₃ | 1.11 | s | | O = CCH₃ | 22.2 |
| | | | | | O = CCH₃ | 169.9 |
| | H-3 | 5.33 | dd | 9.5, 9.2 | C-3 | 72.1 |
| | H-4 | 3.94 | dd | 9.3, 9.2 | C-4 | 74.4 |
| | H-5 | 3.56 | m | | C-5 | 72.7 |
| | H-6x | 4.37 | m | | C-6 | 62.1 |
| | H-6y | | | | | |
| Galβ1-4GlcNAcβ1-3Galβ1-4Glc | H-1 | 4.39 | d | 8.0 | C-1 | 99.9 |
| | H-2 | 5.03 | dd | 8.0, 7.7 | C-2 | 73.4 |
| | H-3 | 4.14 | m | | C-3 | 75.5 |
| | H-4 | 4.00 | dd | 5.6, 2.2 | C-4 | 73.1 |
| | >C(CH₃)₂ | 1.19 | s | | >C(CH₃)₂ | 26.1 |
| | >C(CH₃)₂ | 1.41 | s | | >C(CH₃)₂ | 27.3 |
| | | | | | >C(CH₃)₂ | 110.7 |
| | H-5 | 3.54 | m | | C-5 | 72.0 |
| | H-6x | 3.80 | m | | C-6 | 61.9 |
| | H-6y | 3.00 | m | | | |

### Example 17

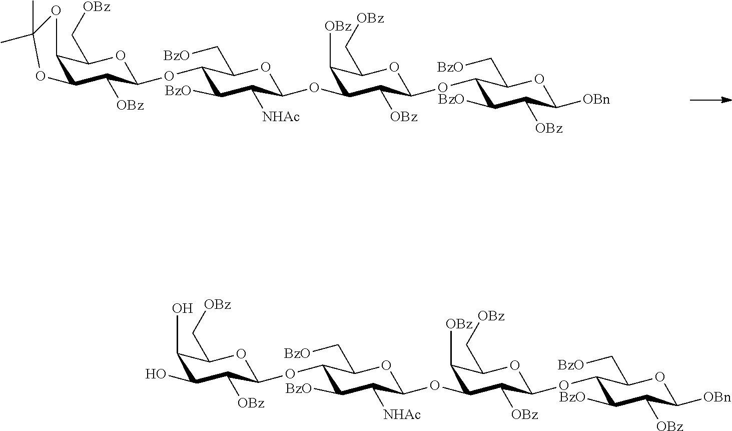

To a solution of compound according to example 16 (11.29 g) in dichloromethane 35% perchloric acid solution (2.3 ml) was added at room temperature under vigorous stirring. After 1 hour the mixture was diluted with dichloromethane (75 ml), washed with sat. sodium bicarbonate and dried. After removal of the solvent the title compound was obtained as a white solid (10.3 g).

LC-MS (ES+): m/z 926.4 [M+2Li]²⁺

¹H and ¹³C resonance assignments in CDCl₃, 30° C., 400 MHz (protecting groups are not detailed in the table):

| Ring | Proton | Shift (ppm) | Mult. | J (Hz) | Carbon | Shift (ppm) |
|---|---|---|---|---|---|---|
| Galβ1-4GlcNAcβ1-3Galβ1-4Glc | H-1 | 4.62 | d | 7.9 | C-1 | 99.0 |
| | CH_{2X}-Ph | 4.76 | d | 12.0 | CH₂-Ph | 70.4 |
| | CH_{2Y}-Ph | 4.53 | d | 12.0 | | |
| | H-2 | 5.42 | dd | 10.0, 7.9 | C-2 | 71.5 |
| | H-3 | 5.59 | dd | 10.0, 9.0 | C-3 | 72.5 |
| | H-4 | 3.97 | m | | C-4 | 75.6 |
| | H-5 | 3.71 | m | | C-5 | 73.1 |
| | H-6x | 4.44 | m | | C-6 | 63.0 |
| | H-6y | | | | | |
| Galβ1-4GlcNAcβ1-3Galβ1-4Glc | H-1 | 4.68 | d | 8.0 | C-1 | 100.6 |
| | H-2 | 5.52 | dd | 9.2, 8.0 | C-2 | 71.4 |
| | H-3 | 4.12 | m | | C-3 | 79.7 |
| | H-4 | 5.88 | dd | 4.0, 3.2 | C-4 | 69.5 |
| | H-5 | 3.75 | m | | C-5 | 71.7 |
| | H-6x | 3.90 | m | | C-6 | 62.9 |
| | H-6y | 2.82 | m | | | |
| Galβ1-4GlcNAcβ1-3Galβ1-4Glc | H-1 | 4.89 | d | 8.6 | C-1 | 101.9 |
| | H-2 | 4.01 | m | | C-2 | 53.9 |
| | O = CNH | 6.16 | br s | | | |
| | O = CCH₃ | 1.03 | s | | O = CCH₃ | 22.1 |
| | | | | | O = CCH₃ | 170.1 |
| | H-3 | 5.40 | dd | 10.1, 9.7 | C-3 | 73.5 |
| | H-4 | 3.98 | dd | 9.7, 9.3 | C-4 | 75.8 |
| | H-5 | 3.61 | m | | C-5 | 72.6 |
| | H-6x | 4.40 | m | | C-6 | 61.9 |
| | H-6y | | | | | |
| Galβ1-4GlcNAcβ1-3Galβ1-4Glc | H-1 | 4.32 | d | 8.0 | C-1 | 101.0 |
| | H-2 | 5.12 | dd | 9.5, 8.0 | C-2 | 73.7 |
| | H-3 | 3.56 | m | | C-3 | 71.9 |
| | OH | 3.18 | br s | | | |
| | H-4 | 3.72 | m | 3.0 | C-4 | 68.3 |
| | OH | 3.18 | br s | | | |
| | H-5 | 3.26 | dd | | C-5 | 72.6 |
| | H-6x | 3.96 | m | | C-6 | 61.9 |
| | H-6y | 3.39 | m | | | |

### Example 18

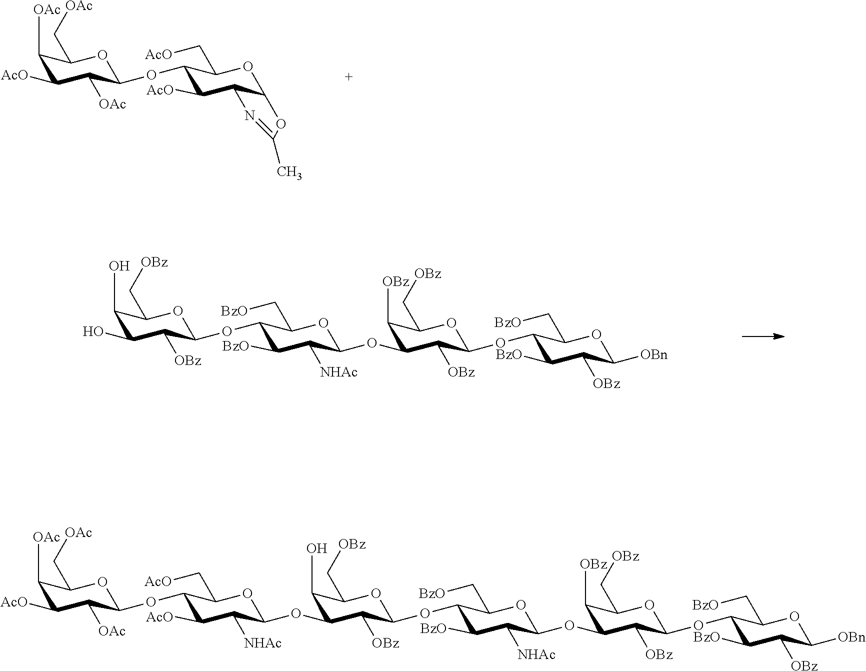

To a solution of the compound according to example 17 (5.0 g) in dichloromethane (6 ml) and toluene (24 ml) heated up to 75° C. BF₃-etherate (67 μl) was added followed by the slow addition of the donor (3.05 g) in toluene (9 ml) at the same temperature in 23 hours. After cooling to room temperature sat. sodium bicarbonate solution was added (5 ml) and the two-phase system was stirred for 5 min. Then ethyl acetate (10 ml) and water (10 ml) were added and the phases were separated, the organic phase was washed with brine and water, and dried. The residue, after removal of the solvents, was purified on a column of silica yielding a white solid (4.55 g).

LC-MS (ES+): m/z 1251 [M+2Na]²⁺, 1240 [M+H+Na]²⁺

### Example 19

### 1-O-benzyl-β-para-LNnH

To a solution of the compound according to example 18 (11.0 g) in methanol (110 ml) 2 mM sodium methoxide solution (1 ml) was added at room temperature. After 14 hours acetic acid (120 μl) was added and the solvent was removed under vacuum. The residue was taken up in water (32 ml) and tetrahydrofuran (65 ml) and treated with 1M NaOH-solution (6.5 ml) at room temperature for 24 hours. After neutralizing with acetic acid (100 μl) the two-phase emulsion was concentrated to 15 ml and diluted with water (10 ml) and methanol (180 ml). A jelly material was formed which was filtered off. The title compound was obtained as a white solid after drying (3.4 g).

LC-MS (ES+): m/z 1169.5 [M+Li]⁺

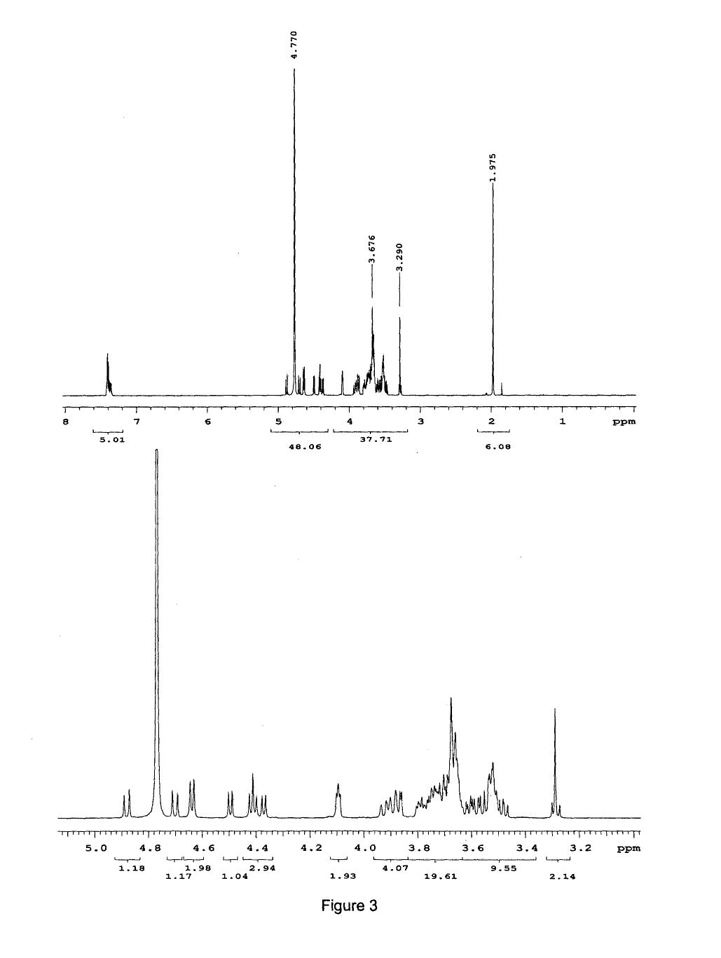

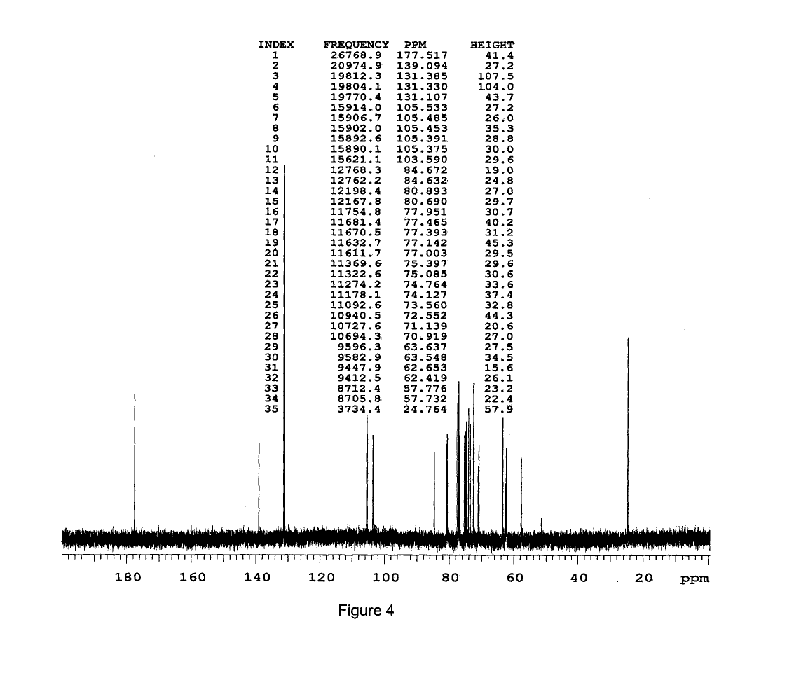

NMR: see FIGS. 3 and 4.

### Example 20

### LNT

1-O-benzyl-β-LNT (5 g) was suspended in water (20 ml) and the pH was adjusted to 5.8 by addition of 1M aq. HCl. Palladium on charcoal (10%, 0.5 g) was added and the reaction flask was evacuated and then saturated with H₂ (4 bar). The reaction temperature was set to 50° C. and after stirring for 1.5 hour the temperature was allowed to reach RT, the catalyst was removed by filtration and water was used for washing (10 ml). The filtrate was concentrated to dryness and 3.46 g (78%) of white solid was obtained.

### Example 21

### LNnH

1-O-benzyl-3-LNnH (10.44 g) was dissolved in water (50 ml) and tetrahydrofuran (50 ml). Palladium on charcoal (10%, 1.04 g) was added and the reaction flask was evacuated and then saturated with H₂. The mixture was stirred for 16 hours at room temperature, then the catalyst was removed by filtration and water was used for washing. The filtrate was concentrated to dryness and 8.06 g (84%) of white solid was obtained.

### Example 22

### para-LNnH

1-O-benzyl-f-para-LNnH (8.23 g) was dissolved in water (100 ml). Palladium on charcoal (10%, 0.90 g,) was added and the reaction flask was evacuated and then saturated with H₂. The mixture was stirred for 16 hours at room temperature, then the catalyst was removed by filtration and water was used for washing. The filtrate was concentrated to dryness and 6.39 g (84%) of white solid was obtained.

1. A method for making a precursor of an HMO core structure comprising a step of reacting a disaccharide glucosamine donor of formula 1 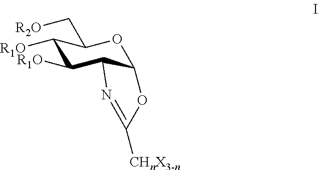
wherein X is a halogen atom selected from the group consisting of F, Cl, Br and I, n is 0, 1, 2 or 3, and one of the R₁-groups is a residue of formula A 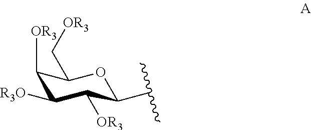
and the other R₁-group is acyl, and R₂ and R₃ are independently acyl, with an acceptor of formula 2 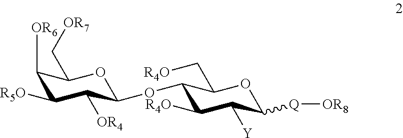
wherein R₄ is acyl, R₅ is selected from the group consisting of H, a peracylated N-acetyllactosaminyl residue and a peracylated lacto-N-biosyl residue, R₆ is H or acyl, R₇ is selected from the group consisting of H, acyl, acetal type groups, silyl and a peracylated N-acetyllactosaminyl residue optionally substituted with 1 or 2 moieties selected from a peracylated N-acetyllactosaminyl group and a lacto-N-biosyl group, R₈ is a group removable by hydrogenolysis, Y is —OR₄ or acetylamino optionally substituted by a halogen atom, Q is a bond when Y is —R₄ and Q is a carbohydrate linker comprising a peracylated lactose moiety optionally substituted either with a peracylated N-acetyllactosaminyl residue or a peracylated lacto-N-biosyl residue when Y is an acetylamino optionally substituted by a halogen atom, provided that at least one of R₅ and R₇ is H,
in the presence of a boron halogenide promoter.

2. The method according to claim 1 which produces a compound of formula 3 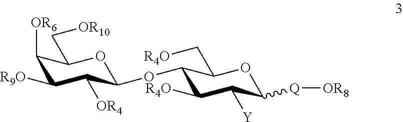
wherein R₄, R₆, R₈, Y and Q are as defined in claim 1, R₉ is selected from the group consisting of a residue of formula B, a peracylated N-acetyllactosaminyl residue and a peracylated lacto-N-biosyl residue, and R₁₀ is selected from the group consisting of a residue of formula B, acyl, acetal type groups, silyl and a peracylated N-acetyllactosaminyl residue optionally substituted with 1 or 2 moieties selected from a peracylated N-acetyllactosaminyl group and a lacto-N-biosyl group, provided that at least one of R₉ and R₁₀ is a residue of formula B 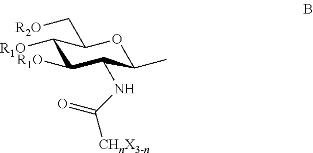
wherein R₁, R₂, X and n are as defined in claim 1.

3. The method according to claim 1, wherein the boron halogenide promoter is boron trifluoride, particularly boron trifluoride etherate.

4. The method according to claim 1, wherein n is 3 and Y is —NHCOCH₃.

5. The method according to claim 4, wherein the acceptor is of formula 2A 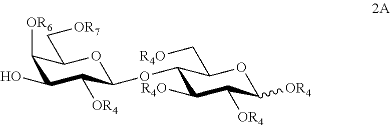
wherein R₄ is acyl, R₆ is H or acyl, R₇ is selected from the group consisting of acyl, acetal type groups and silyl, and R₈ is a group removable by hydrogenolysis,
and the precursor of an HMO core structure is an LNT or LNnT precursor of formula 4 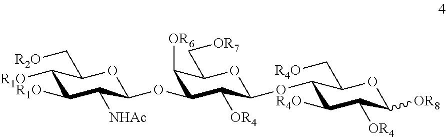
wherein R₁ and R₂ are as defined in claim 1, and R₄, R₆, R₇ and R₈ are as defined above.

6. The method according to claim 5 to obtain an LNnT precursor of formula 4A 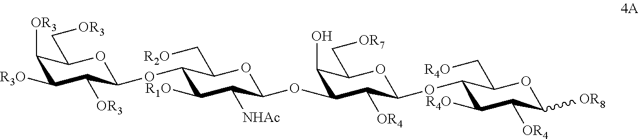
wherein R₁, R₂, R₄, R₆, R₇ and R₈ are defined in claim 5.

7. The method according to claim 5 to obtain an LNT precursor of formula 4B 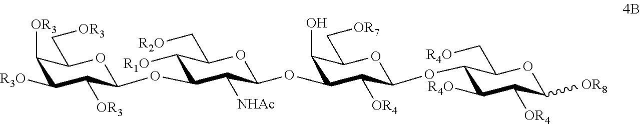
wherein R₁, R₂, R₄, R₆, R₇ and R₈ are defined in claim 5.

8. The method according to claim 4, wherein the acceptor is of formula 2B 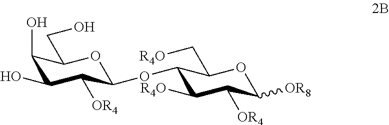
wherein R₄ is acyl and R₈ is a group removable by hydrogenolysis.

9. The method according to claim 4, wherein the acceptor is of formula 2C or 2D 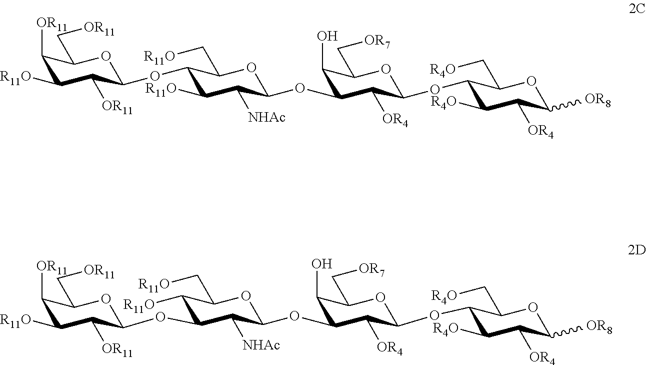
wherein R₄ and R₁₁ are independently acyl, and R₈ is a group removable by hydrogenolysis.

10. The method according to claim 4, wherein the acceptor is of formula 2E 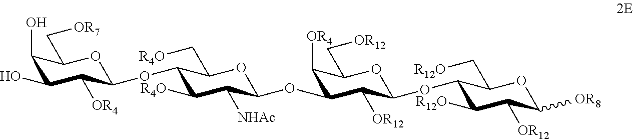
wherein R₄ is acyl, R₇ is selected from the group consisting of acyl, acetal type groups and silyl, R₈ is a group removable by hydrogenolysis, and R₁₂ is acyl.

11. The method according to claim 1, wherein R₄ is a low-migrating acyl group.

12. The method according to claim 11, wherein R₄ is a linear or branched chain alkanoyl group of 4 or more carbon atoms, or an unsubstituted or substituted benzoyl or naphthoyl group.

13. The method according to claim 11, wherein the R₁-group not being the residue A, R₂ and R₃ are identical and are acetyl or benzoyl, R₈ is benzyl, and —OR₈ is in β-orientation.

14. The method according to claim 1 comprising a further step of de-O-acetylating the compound of formula 3 to obtain an R₈-glycoside of an HMO core structure of formula 7 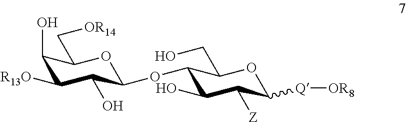
wherein R₈ is a group removable by hydrogenolysis, Z is —OH or acetylamino optionally substituted by a halogen atom, Q′ is a bond when Y is —OH and Q′ is a carbohydrate linker comprising a lactose moiety optionally substituted with an N-acetyllactosaminyl residue or a lacto-N-biosyl residue when Z is an acetylamino optionally substituted by a halogen atom, R₁₃ is selected from the group consisting of a residue of formula C, an N-acetyllactosaminyl residue and a lacto-N-biosyl residue, R₁₄ is selected from the group consisting of H, a residue of formula C, and an N-acetyllactosaminyl residue optionally substituted with 1 or 2 moieties selected from an N-acetyllactosaminyl group and a lacto-N-biosyl group, provided that at least one of R₁₃ and R₁₄ is a residue of formula C 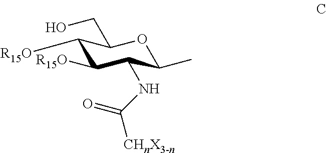
wherein one of the R₁₅-groups is a β-D-galactopyranosyl group and the other R₁₅-group is H, X is a halogen atom selected from the group consisting of F, Cl, Br and I, and n is 0, 1, 2 or 3.

15. The method according to claim 14, wherein the compound of formula 7 is 1-O-benzyl LNT, 1-O-benzyl LNnT, 1-O-benzyl LNnH or 1-O-benzyl para-LNnH.

16. The method according to claim 1 comprising a further step of catalytic hydrogenolysis to obtain an HMO core structure.

17. A compound of formula 3′ 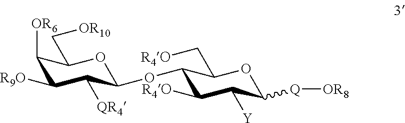
wherein R₄′ is a low-migrating acyl group, R₆ is H or acyl, R₈ is a group removable by hydrogenolysis, Y is —OR₄′ or acetylamino optionally substituted by a halogen atom, Q is a bond when Y is —OR₄′ and Q is a carbohydrate linker comprising a peracylated lactose moiety optionally substituted with either a peracylated N-acetyllactosaminyl residue or a peracylated lacto-N-biosyl residue when Y is an acetylamino optionally substituted by a halogen atom, R₉ is selected from the group consisting of a residue of formula B, a peracylated N-acetyllactosaminyl residue and a peracylated lacto-N-biosyl residue and R₁₀ is selected from the group consisting of a residue of formula B, acyl, acetal type groups, silyl and a peracylated N-acetyllactosaminyl residue optionally substituted with 1 or 2 moieties selected from a peracylated N-acetyllactosaminyl group or a peracylated lacto-N-biosyl group, provided that at least one of R₉ and R₁₀ is a residue of formula B 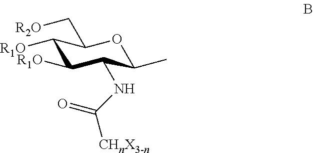
wherein X is a halogen atom selected from the group consisting of F, Cl, Br and I, n is 0, 1, 2 or 3, and one of the R₁-groups is a residue of formula A 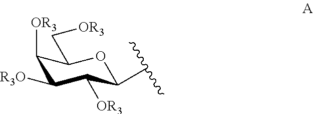
and the other R₁-group is acyl, R₂ and R₃ are independently acyl.

18. The compound of claim 17, wherein R_{4′} is a linear or branched chain alkanoyl group of 4 or more carbon atoms or an unsubstituted or substituted benzoyl or naphthoyl group.

19. The compound of claim 17, wherein the R₁-groups not being the residue A, R₂ and R₃ are identical and are acetyl or benzoyl, R₅ is H, R₆ is benzyl, and —OR₆ is in β-orientation.

20. A compound of formula 2B 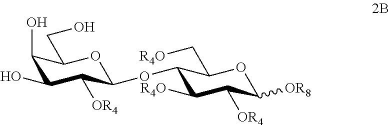
wherein R₄ is acyl and R₈ is a group removable by hydrogenolysis.
